SEQUENCE LISTING (15 sequences)

SEQ ID NO: 1 - protein, 11 aa, Artificial Sequence
  Synthetic polypeptide
ARAMGYPGASY

SEQ ID NO: 2 - protein, 11 aa, Artificial Sequence
  Synthetic polypeptide
ARAFGYPIYSY

SEQ ID NO: 3 - protein, 11 aa, Artificial Sequence
  Synthetic polypeptide
YYPGAYPRRYR

SEQ ID NO: 4 - protein, 11 aa, Artificial Sequence
  Synthetic polypeptide
AMAMGYPRASY

SEQ ID NO: 5 - protein, 11 aa, Artificial Sequence
  Synthetic polypeptide
AMARGYPWYSY

SEQ ID NO: 6 - protein, 11 aa, Artificial Sequence
  Synthetic polypeptide
SYMRAYPMQIP

SEQ ID NO: 7 - protein, 11 aa, Artificial Sequence
  Synthetic polypeptide
SYMRAYPMQMP

SEQ ID NO: 8 - protein, 11 aa, Artificial Sequence
  Synthetic polypeptide
YYPGAYPMRFR

SEQ ID NO: 9 - protein, 7 aa, Artificial Sequence
  Synthetic polypeptide
AYPRRYR

SEQ ID NO: 10 - protein, 250 aa, Homo sapiens [sequence omitted]

SEQ ID NO: 11 - protein, 100 aa, Homo sapiens
MADNFSLHDALSGSGNPNPQGWPGAWGNQPAGAGGYPGASYPGAYPGQAPPGAYPGQAPPGAYPGAPGAYPGAPAPGVYPGPPSGPGAYPSSGQPSATGA

SEQ ID NO: 12 - protein, 150 aa, Homo sapiens
YPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 13 - protein, 16 aa, Artificial Sequence
  Synthetic polypeptide
ANTPCGPYTHDCPVKR

SEQ ID NO: 14 - protein, 16 aa, Artificial Sequence
  Synthetic polypeptide
PTHVTCKYCPAGNRDP

SEQ ID NO: 15 - protein, 4 aa, Artificial Sequence
  Synthetic polypeptide
PGAY

(19) United States
(12) Patent Application Publication
(10) Pub. No.: US 2023/0273213 A1
(43) Pub. Date: Aug. 31, 2023

(54) PEPTIDE DESIGN AND GALECTIN-3 INHIBITORS

(71) Applicant: City of Hope, Duarte, CA (US)

(72) Inventors: Nora Heisterkamp, Monrovia, CA (US); Supriyo Bhattacharya, Duarte, CA (US)

(21) Appl. No.: 18/017,841

(22) PCT Filed: Aug. 2, 2021
(86) PCT No.: PCT/US2021/044164

§ 371 Date: Jan. 24, 2023

Related U.S. Application Data
(60) Provisional application No. 63/059,305, filed on Jul. 31, 2020.

(51) Int. Cl. G01N 33/574; G01R 33/46; G16B 15/20; A61K 38/17

(52) U.S. Cl. CPC G01N 33/57484; G01R 33/46; G16B 15/20; A61K 38/1732; G01N 2333/4724

(57) ABSTRACT

Provided herein are, inter alia, methods and systems for the in silico design of peptide inhibitors for proteins comprising disordered domains; Galectin-3 inhibitors; and methods for treating and detecting diseases that overexpress or inappropriately express Galectin-3.

### CROSS-REFERENCE TO RELATED APPLICATIONS

This application claims priority to U.S. Application No. 63/059,305 filed Jul. 31, 2020, the disclosure of which is incorporated by reference herein in its entirety.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-772001WO_SL_ST25.txt, created Jul. 21, 2021, 7,230 bytes, machine format IBM-PC, MS Windows operating system, is hereby incorporated by reference.

### BACKGROUND

Many multifunctional proteins contain one or more domains with no clearly-defined 3-dimensional structure (1). Although such intrinsically disordered regions (IDRs) are generally small (e.g., less than 100 amino acids) they are surprisingly abundant and have important functions in the proteins that contain them: Afafasyeva et al (2), who analyzed such structures, identified 6600 human proteins containing IDRs. The lack of a higher order structure in IDRs allows such domains to be extremely flexible, and most IDR-containing proteins are known to functionally engage in protein and RNA/DNA interactions (2).

Galectin-3 can be described as a carbohydrate-binding protein but this does not adequately capture its highly diverse cellular roles: it has been recovered at many different subcellular locations including the nucleus, the cytoplasm (at the ER-mitochondrial interface) (3); in spindle poles (4) associated with lysosomes and autosomes (5); in membrane-less cytoplasmic ribonucleotide-protein (RNP) particles (6, 7) as well as bound to the cell surface, and secreted into the extracellular space including peripheral blood. More than 300 proteins can form complexes with Galectin-3 in hematopoietic stem cells and peripheral blood mononuclear cells (8), and Galectin-3 has been implicated in numerous pathologies ranging from heart disease and diabetes to cancer (9, 10).

The N-terminal end of Galectin-3 contains an intrinsically disordered region of around 80 amino acids, with the C-terminal domain (CTD) consisting of two faces, the F-face and the S-face. The S-face is the moiety that recognizes and binds to specific glycoproteins and includes the carbohydrate-recognition/binding domain (CRD). The function of the CRD has been studied in most detail on the surface of cells, which are covered by a dense layer of carbohydrate-containing biomolecules including glycoproteins and glycolipids. At that location, extracellular Galectin-3 regulates signal transduction strength of glycoprotein receptors through its multimerization and crosslinking activity, resulting in intermolecular and intercellular lattice complex formation (11).

Increased Galectin-3 expression correlates with many different disease states including inflammation and cancer, but a direct cause-effect relationship has also been demonstrated for some of these using knockout models. Thus, the ability to inhibit the protein is viewed as an important goal with the ultimate objective to therapeutically target Galectin-3 in different diseases (12, 13). To this end, efforts have mainly focused on the CRD: because the structure of the CTD has been determined, and the interactions of the CRD with glycans have been well-described, many carbomimetics that will interfere with the ability of Galectin-3 to bind to glycoprotein targets have been reported (14, 15). TD139 is a Galectin-3 inhibitor in this category (16) that is being tested as an inhaled drug in clinical trials for idiopathic fibrosis (17). However, some of such compounds may have unfavorable pharmacokinetic properties (18) and, as reviewed in (19), there are currently few examples of glycan-directed therapies that have transitioned to clinical use. This may be also due to challenges relating to shallow solvent-exposed binding surfaces, lack of many hydrophobic residues for ligand contact and low residence time of the bound inhibitors when lectins bind to their carbohydrates.

The N-terminal domain of Galectin-3 also appears to have a critically important contribution to its function (20) and was recently shown to mediate protein multimerization (21). Removal of this domain yields a CTD Galectin-3 protein with dominant negative activity (22-24). The CTD of Galectin-3 also contains a domain that is not the main site of direct C-terminal/binding called the F-face. Ippel et al (25) showed that the NTD interacts transiently with the CTD F-face and characterized this interaction in more detail. Moreover, Lin et al 2017 (26) reported that the disordered N-terminal domain including amino acids 20-100 forms a fuzzy complex with n-strand regions of the F-face. Importantly, the NTD mediates liquid-liquid phase separation of Galectin-3 (21, 27) which could explain its contribution to forming membrane-less structures such as cytoplasmic RNP.

Galectin-3 specifically recognizes the Gal-GlcNAc (poly-LacNAc) branches of N-glycans on glycoproteins to carry out its function. However, inhibition of binding of the lectin domain to the glycan target is problematic due to the shallow solvent exposed binding surface, lack of many hydrophobic residues for ligand contact, and low residence time of the bound inhibitors. So far, none of the glycomimetic compounds targeting Galectin-3 have shown potent activity in tissue culture models. There is a need in the art for methods for developing inhibitors of proteins that contain a disordered domain (e.g., the NTD of Galectin-3) and for drugs that inhibit Galectin-3. The disclosure is directed to these, as well as other, important ends.

### BRIEF SUMMARY

The disclosure provides method of identifying an amino acid within a disordered domain of a protein that binds to an ordered domain of a protein with the ordered domain either located in the same protein or in a different protein, the method comprising: (i) in silico, performing an enhanced sampling of a disordered domain of a protein binding to an ordered domain of the same protein or an ordered domain of a different protein thereby obtaining an ensemble of conformations, wherein each conformation in the ensemble comprises the disordered domain bound to the ordered domain; (ii) identifying a first set of structural conformations from the ensemble of conformations that satisfy the experimental structural NMR data of the protein; and (iii) identifying a first amino acid within the first set of structural conformations, wherein the first amino acid is within the disordered domain of the protein that binds to the ordered domain of the same protein or binds to the ordered domain of the different protein. In aspects, the methods further comprise (iv) clustering the first set of structural conformations by structural similarity to identify template peptides. In aspects, the methods further comprise (a) designing a plurality of template peptides that bind in silico to a first amino acid in the ordered domain based at least in part on the first set of structural conformations; (b) in silico, mutating each residue of each of the plurality of template peptides thereby producing a plurality of mutant peptides; (c) selecting a set of candidate peptides from the plurality of mutant peptides based on in silico binding; (d) synthesizing each of the set of candidate peptides thereby producing a set of synthesized candidate peptides; and (e) experimentally measuring the effect of each of the synthesized candidate peptides on the protein.

The disclosure provides Galectin-3 inhibitors, such as small molecules and the peptides, and methods of treating diseases mediated by overexpression or inappropriate expression of Galectin-3.

These and other embodiments and aspects of the disclosure are provided herein.

### BRIEF DESCRIPTION OF THE DRAWINGS

FIGS. 1A-1D provide information on the structure of Galectin-3. FIG. 1A is a schematic showing the inhibition of the NTD-CTD interaction in Galectin-3 using synthetic inhibitors. In particular, Peptide-3 inhibits the IDR from contacting the shallow pocket in the F-face of the CTD which can be located on the same or on a different Galectin-3 molecule. FIG. 1B provides a comparison of the experimental chemical shift differences to those calculated from the AMD structural ensemble. The left side of FIG. 1C shows that conformations from the AMD simulations are clustered by structural similarity; for each cluster, the number of NTD-CTD contacts and root mean square deviation from experimental chemical shift differences are plotted against each other; clusters that show high NTD-CTD contacts and low RMSD with experimental chemical shift differences are circled. The right side of FIG. 1C and the left side of FIG. 1D shows the two major NTD structural ensembles that agree with the experimental NMR data, where the NTD residues that form the major contacts with the CTD in these ensembles (Y36 and Y45) are highlighted. The right side of FIG. 1D provides an expanded view of the interface between the NTD and the CTD showing the major CTD residues in contact with the NTD. On the right side, bottom panel, of FIG. 1D, amino acid residue Y45 is in the NTD; amino acid residues with strong NMR peak intensity in the CTD are V202, K210, and A216, and amino acid residues with medium NMR peak intensity in the CTD are F192, F198, K199, L203, V204, D215, H217, Q220, and L219.

FIGS. 2A-2D show the results and method used to identify the Galectin-3 inhibitor peptide candidates. FIG. 2A shows the steps in generating the initial peptide templates. FIG. 2B shows the steps in designing the top peptide candidates starting from the initial templates. FIG. 2C shows the protein-peptide interaction energies of the top 8 candidate peptides. FIG. 2D shows the duration of the peptide binding to the CTD as observed in molecular dynamics simulations.

FIGS. 3A-3H show that peptide 3 inhibits agglutination of human leukemia cells mediated by Galectin-3. Representative brightfield images of suspensions of pediatric pre-B acute lymphoblastic leukemia LAX56 cells. Cells were incubated for 2 hours (FIG. 3A) with no added protein; (FIG. 3B) with control GST; or (FIGS. 3C-3G) with GST-Gal3. FIG. 3D: Addition of 100 μM TD139 Galectin-3 inhibitor. FIGS. 3E-3G: Peptides as indicated were added together with GST or GST-Gal3. Similar results were obtained with two independently generated batches of GST-Gal3; representative images are shown. FIG. 3H: Quantification of agglutination expressed as the number of cell aggregates.

FIGS. 4A-4B show, via site-directed mutagenesis of Galectin-3, that combined L131 and L203 are essential for agglutination. FIG. 4A: Brightfield images of LAX56 cells incubated with the recombinant fusion proteins indicated to the right. Peptides added together with the recombinant proteins are noted above the figure. FIG. 4B: Quantitation of cellular aggregation under the indicated conditions.

FIGS. 5A-5C show residues in the Galectin-3 CTD domain with significant chemical shift perturbations through interaction with P3 peptide-3. FIG. 5A: The ¹H-¹⁵N HSQC spectrum in blue was acquired on free ¹⁵N labeled Galectin-3 CTD. The spectrum in red was acquired on the complex between ¹⁵N labeled CTD and peptide-3 with molar ratio of 100:1 between P3 peptide and ¹⁵N labeled CTD. Some residues with significant chemical shift perturbations are labeled, including two side chains from Q201 and N214. FIG. 5B: Selected overlay of ¹H-¹⁵N HSQC spectrum region of ¹⁵N labeled CTD versus titration of P3 peptide. Residues with notable chemical shift changes are labeled together with the cross peak moving direction, as indicated by the arrow, with increased concentration of P3 peptide. Spectrum in black, free CTD; spectra in red, green, blue and magenta: molar ratio of 20:1, 40:1, 60:1 and 100:1 between P3 peptide and Gal3 CTD, respectively. FIG. 5C: Chemical shift perturbation of ¹⁵N-CTD in complex with P3 peptide versus primary sequence of residues 117-250. The chemical shift changes between free ¹⁵N-CTD and in complex with 100-fold molar excess of P3 peptide are indicated. The thin horizontal line indicates the limit above which values of ¹⁵N-CTD in complex with P3 peptide are two times the RMSD of the CSP of free of ¹⁵N-CTD. V138 and E205 are color-coded in blue since they shifted apart in complex from the overlaid cross peak in free ¹⁵N-CRT, and their CSP values could be swapped.

FIG. 6 is a contact heatmap of conformations with high BME weights. CTD residues with significant experimental NMR shifts are as indicated with red font below the heatmap. NTD residues making frequent CTD contacts include A2, A49, A53, A69, D3, F5, G108, G112, G43, G47, G52, G68, G72, H8, P106, P71, Q20, Q48, S84, T98, V78, W22, Y101, Y41, Y45, Y54, Y70 and Y79.

FIGS. 7A-7B show a schematic of Galectin-3. FIG. 7A: The CTD with the β-sheets of the F-face as indicated. Amino acids present in each β sheet of the F-face are indicated. Residues making strong contact with peptide-3 are bold; L131 and L203 residues are underlined. FIG. 7B: The binding mode of peptide 3 to Galectin-3 CTD, as predicted from the MD simulation. The CTD residues within 5 Å of the bound peptide are highlighted as sticks. Residues that show significant chemical shifts are highlighted in red. The peptide is shown as a magenta cartoon. The tyrosine corresponding to the central PGAY (SEQ ID NO:15) motif is displayed as a stick.

FIGS. 8A-8C show contributions of the CTD residues in binding of NTD. FIGS. 8A-8B: The interaction energies of the top CTD residues with the NTD in two major clusters derived from the AMD simulations in which either Y36 (FIG. 8A) or Y45 (FIG. 8B) of the NTD inserts into the F face. The bars representing the highest energy contribution are colored red. The residues of which mutations resulted in significant loss of agglutination are highlighted by red boxes. FIG. 8C: Zoomed-in view of the binding pocket of the NTD in the CTD in which Y45 inserts into the F face. The CTD residues showing the highest contribution to NTD binding are highlighted in red. Y45 is colored green. The CTD and the NTD are shown as grey and orange cartoons respectively. The hydrogen bond between the —OH group of Y45 and H217 is shown as a dotted red line.

### DETAILED DESCRIPTION

### Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The terms “protein,” “peptide,” and “polypeptide” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are modified, e.g., hydroxyproline, γ-carboxyglutamate, O-phosphoserine, or have O-GlcNAc or other glycans attached. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. Amino acids may be referred to herein by either their commonly known three letter symbols or by the one-letter symbols recommended by the IUPAC-IUB Biochemical Nomenclature Commission. Nucleotides may be referred to by their commonly accepted single-letter codes.

“Ordered protein domain” or “ordered domain of a protein” is a domain in a protein that has a fixed or ordered three-dimensional structure. In aspects, “ordered domain of a protein” refers to a conserved part of a given protein sequence and structure (e.g., tertiary) that can function and exist independently of the rest of the protein chain. Ordered domains of a protein can be identified by using, e.g., the National Center for Biotechnology Information (NCBI) website; in particular, the conserved domain annotation under the “refSeq section” of the gene information may be used.

“Disordered protein domain” or “disordered domain of a protein” is a domain in a protein that does not have a fixed or ordered three-dimensional structure. “Intrinsically disordered protein” refers to a protein that does not have a fixed or ordered three-dimensional structure.

A “protein-protein interaction interface,” “protein-protein interface,” or an “interface” includes the “contact” residues (one or more amino acids and/or other non-amino acid residues such as carbohydrate groups, NADH, biotin, FAD or heme group) in a first protein domain that interact with one or more “contact” residues (one or more amino acids and/or other non-amino acid groups) in the interface of a second protein domain. As used herein, a “contact residue” refers to any amino acid and/or non-amino acid residue from one domain that interacts with another amino acid and/or non-amino acid residue from a different domain by van der Waals forces, hydrogen bonds, water-mediated hydrogen bonds, salt bridges or other electrostatic forces, attractive interactions between aromatic side chains, the formation of disulfide bonds, or other forces known to one skilled in the art. Typically, the distance between alpha carbons of two interacting contact amino acid residues in the interaction interface is no greater than 12 angstroms. In aspects, the first protein domain is a disordered protein domain and the second protein domain is an ordered protein domain.

“Conformational ensembles” or “structural ensembles” are computational models that attempt to describe the structure of a disordered domain of a protein or an intrinsically unstructured protein (i.e., flexible proteins or flexible protein domains that lack a stable tertiary structure and that cannot be described with a single structural representation.

“In silico” means performed on a computer or by a computer simulation.

The term “enhanced sampling” refers to molecular dynamics, enhanced molecular dynamics, Monte Carlo, or any other conformational sample technique.

“Enhanced molecular dynamics simulation” refers to computer simulation methods for analyzing the physical movements of atoms and molecules. The atoms and molecules are allowed to interact for a fixed period of time, giving a view of the dynamic evolution of the system. Exemplary enhanced molecular dynamic simulations include accelerated molecular dynamic simulations (e.g., Hamelberg et al, Journal of Chemical Physics, 120(24):11919-11929 (2004)), replica exchange molecular dynamic simulation, metadynamics simulation, temperature cool walking, and generalized simulated annealing

“Candidate peptide” refers to a peptide of interest that is predicted to modulate (e.g., inhibit) a target protein (e.g., Galectin-3). A “synthesized candidate peptide” refers to a candidate peptide that has been manufactured (e.g., synthesized by chemical and/or biological processes).

The term “Galectin-3” or “Gal3” as used herein includes any of the recombinant or naturally-occurring forms of Galectin-3 or variants or homologs thereof that maintain Galectin-3 activity (e.g. within at least 50%, 80%, 90%, 95%, 96%, 97%, 98%, 99% or 100% activity compared to Galectin-3). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Galectin-3 protein. In aspects, the Galectin-3 protein is substantially identical to the protein identified by SEQ ID NO:10, UniProt reference number P17931, or a variant or homolog having substantial identity thereto.

The term “disordered N-terminal domain of Galectin-3” or “N-terminal domain of Galectin-3” refers to SEQ ID NO:11, or variants or homologs thereof having at least 90%, 95%, 96%, 97%, 98%, 99% or 100% amino acid sequence identity across the whole sequence or a portion of the sequence.

The term “C-terminal domain of Galectin-3” or “ordered C-terminal domain of Galectin-3” or “CTD” refers to SEQ ID NO:12, or variants or homologs thereof having at least 90%, 95%, 96%, 97%, 98%, 99% or 100% amino acid sequence identity across the whole sequence or a portion of the sequence. The CTD is composed of eleven β-strands (or sheets) running in antiparallel fashion, with six of the β-strands (i.e., β1, β10, β3, β4, β5, β6) defining the carbohydrate recognition/binding domain (S-face) and five of the β-strands (i.e., β11, β2, β7, β8, β9) defining the F-face. The CTD also includes the amino acid residues in the loops between (or connecting) the β-strands. Thus, the F-face may be further defined to include the loop between β8 and β9, the loop between β7 and β8, the loop between β1 and β2, and the loop between β2 and β3.

The term “inhibitor,” “inhibition,” “inhibit,” “inhibiting” and the like in reference to a protein-inhibitor interaction means negatively affecting (e.g., decreasing) the activity or function of the protein (e.g., decreasing the activity of Galectin-3) relative to the activity or function of the protein in the absence of the inhibitor. In aspects, inhibition refers to reduction of a disease or symptoms of disease (e.g., cancer). Thus, inhibition includes, at least in part, partially or totally blocking stimulation, decreasing, preventing, or delaying activation, or inactivating, desensitizing, or down-regulating signal transduction or enzymatic activity or the amount of a protein (e.g., a Galectin-3 protein). Similarly an “inhibitor” is a compound or protein that inhibits a receptor or a protein, e.g., by binding, partially or totally blocking, decreasing, preventing, delaying, inactivating, desensitizing, or down-regulating activity (e.g., Galectin-3 protein activity).

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a composition is substantially purified.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below or by manual alignment and visual inspection (e.g., http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are naturally or synthetically modified such as, but not limited to hydroxyproline, γ-carboxyglutamate, and O-phosphoserine, or have O-GlcNAc or other glycans. In aspects, the amino acid side chain is a non-natural amino acid side chain. In aspects, the amino acid side chain is H,

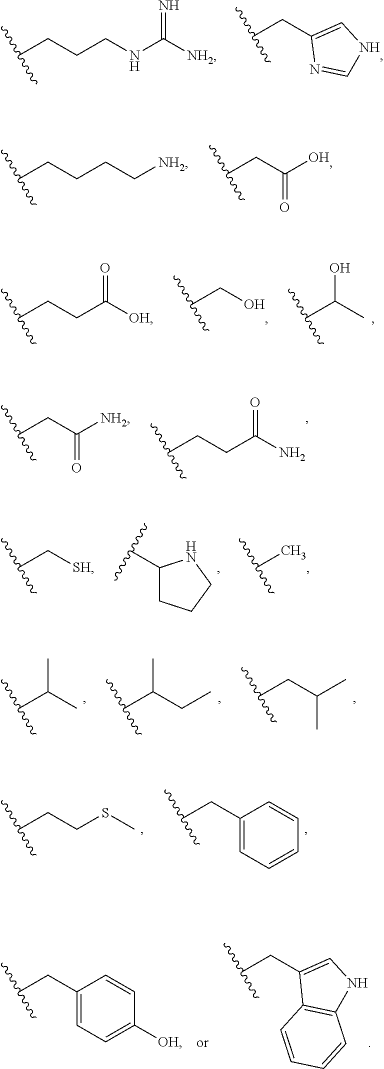

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo-[2.2.1]hept-5-ene-2-carboxylic acid hydrochloride, cis-2-aminocycloheptanecarboxylic acid hydrochloride, cis-6-amino-3-cyclohexene-1-carboxylic acid hydrochloride, cis-2-amino-2-methylcyclohexane-carboxylic acid hydrochloride, cis-2-amino-2-methylcycloopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-homopyr-OH, Boc-(2-indanyl)-Gly-OH, 4-Boc-3-morpholineacetic acid, 4-Boc-3-morpholine acetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH₂)—OH, Boc-Phe(3-NO₂)—OH, Boc-Phe(3,5-F2)-OH, 2-(4-Boc-piperazino)-2-(3,4-dimethoxy-phenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)-acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)-phenyl]acetic acid purum, Boc-β-(2-quinolyl)-Ala-OH, N-Boc-1,2,3,6-tetrahydro-2-pyridine-carboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-penta-fluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, and 4-(hydroxymethyl)-D-phenylalanine.

A “control” sample or value refers to a sample that serves as a reference, usually a known reference, for comparison to a test sample. For example, a test sample can be taken from a patient suspected of having a given disease (cancer) and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. In aspects, a control is a negative control. In aspects, such as embodiments relating to detecting the level of expression, a control comprises the average amount of expression (e.g., protein) of infiltration (e.g., number or percentage of cells in a population of cells) in a population of subjects (e.g., with cancer) or in a healthy or general population. In aspects, the control comprises an average amount (e.g. percentage or number of infiltrating cells or amount of expression) in a population in which the number of subjects (n) is more than 1. In aspects, the control is a standard control. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “overexpression” or “protein overexpression” refers to an increased expression of a protein relative to a control (e.g., relative to a healthy control).

The term “inappropriate expression” or “abnormal expression” refers to protein misfolding, abnormal conformations, mutations in expressed proteins, and also refers to expression at normal levels but at an abnormal and/or inappropriate anatomical location or at an abnormal and/or inappropriate moment in a series of physiological events.

The term “bind” and “bonded” is used in accordance with its plain and ordinary meaning and refers to the association between atoms or molecules. The association can be direct or indirect. For example, atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g., dipole-dipole, dipole-induced dipole), ring stacking (pi effects), hydrophobic interactions and the like).

The term “and/or” means either one or both of two stated possibilities. For example, Y36 and/or Y45 means: (1) Y36; (2) Y45; or (3) Y36 and Y45.

### Peptide Design

Intrinsically disordered regions (IDRs) are common and important functional domains in many proteins. However, IDRs are difficult to target for drug development due to the lack of defined structures which would facilitate the identification of possible drug-binding pockets. Galectin-3 is a carbohydrate-binding protein of which overexpression has been implicated in a wide variety of disorders including cancer and inflammation. Apart from its C-terminal/binding domain (CTD), Galectin-3 also contains a functionally important disordered N-terminal domain (NTD) that contacts the C-terminal domain (CTD) and could be a target for drug development.

To overcome challenges involved in inhibitor design due to lack of structure and the highly dynamic nature of the NTD, we used a novel protocol combining nuclear magnetic resonance data from recombinant Galectin-3 with accelerated molecular dynamics (MD) simulations to identify a shallow pocket in the CTD with which the NTD makes frequent contact. In accordance with this model, a Galectin-3 double mutant of residues L131 and L203 in the CTD lost agglutination ability. In-silico design was used to narrow down candidate inhibitory peptides and experimental testing of only 3 of these yielded one peptide that inhibits the agglutination promoted by wild type Galectin-3. NMR experiments further confirmed that this peptide makes contacts with a non-carbohydrate binding moiety of the CTD. Our results show that it is possible to apply a combination of MD simulations and NMR experiments to precisely predict the binding interface of a disordered domain with a structured domain, and furthermore use this predicted interface for designing inhibitors. This procedure can thus be potentially extended to many other targets in which similar IDR interactions play a vital functional role.

A key step in the peptide design process was to obtain the ensemble of NTD conformations that interacted with the CTD under physiological conditions. Since the NTD is an intrinsically disordered region (IDR), it adopts multiple conformations under physiological conditions and is also highly dynamic. Therefore, methods such as X-ray crystallography that are typically used for determining protein structures are not applicable to IDRs. NMR spectroscopy can give structural information about IDRs in the form of peak intensities for individual residues in the amino acid sequence. However, NMR does not directly provide the 3D structural coordinates of the protein atoms, which are necessary for inhibitor design, unless the NMR data is interpreted using a predetermined protein structural ensemble generated in-silico. To generate the in-silico structural ensemble, MD simulations and Monte Carlo sampling of backbone dihedrals are typically used, but each of these methods suffers from their own deficiencies. Due to the vast protein conformational space, Monte Carlo based methods may not be able to sample all the relevant conformations in reasonable time, whereas all atom MD simulations can only sample conformations that are accessible over a timescale of nanoseconds to low microseconds. IDR conformational transitions may span a timescale of hundreds of microseconds to milliseconds, which are beyond the reach of conventional MD simulations. Thus, it is challenging to generate an IDR structural ensemble using in-silico methods, which will cover the physiological IDR conformations. Thus, the challenges involved in the inhibitor design included: (1) generating a structural ensemble of the NTD-CTD complex using in-silico methods that include the physiological NTD conformations; (2) detecting the physiological NTD conformations from the very large in-silico ensemble using experimental information such as NMR, and (3) accounting for the dynamic nature of the NTD in the inhibitor design protocol; i.e. to be effective, the designed inhibitors should be able to disrupt the interactions of multiple structurally diverse NTD conformations binding to the ordered C-terminal domain.

To address the above challenges, a computational pipeline incorporating state-of-the-art MD simulation methods and in-silico peptide design algorithms was developed. To address the problem of IDR conformational sampling, an enhanced MD method called accelerated MD (AMD) was used (Hamelberg et al., 2004). Using energy rescaling, AMD is capable of accessing timescales in the order of milliseconds, that are beyond the reach of conventional MD. Starting from an initial protein structure (e.g., Galectin-3), the CTD is modeled based on an existing crystal structure and the NTD is modeled as a random polymer chain, thereafter, AMD is used to generate the initial conformational ensemble (e.g., having 50,000 NTD conformations). For each of these conformations, the corresponding chemical shifts are predicted using the software SHIFTX2, for both the full length protein as well as for the CTD alone (Han et al., 2011). The chemical shift differences (CSDs) are then calculated according to the formula:

Δδ ppm=[(Δ¹H)²+(0.25Δ¹⁵N)²]^{1/2}

where Δ¹⁵N and Δ¹H are the chemical shift differences of the ¹⁵N labeled backbone nitrogen and hydrogen atoms between full length and CTD-only Galectin-3. The NTD conformations are clustered by their structural similarity and for each cluster, the root mean square deviation (RMSD) from the experimental NMR CSDs are calculated. The clusters showing low CSD RMSD and a high number of NTD-CTD contacts (e.g., about 1300 conformations) are then selected for further processing.

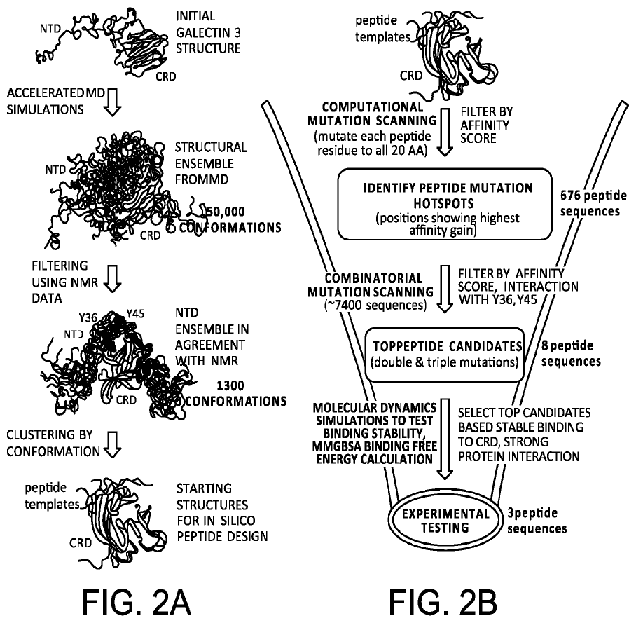

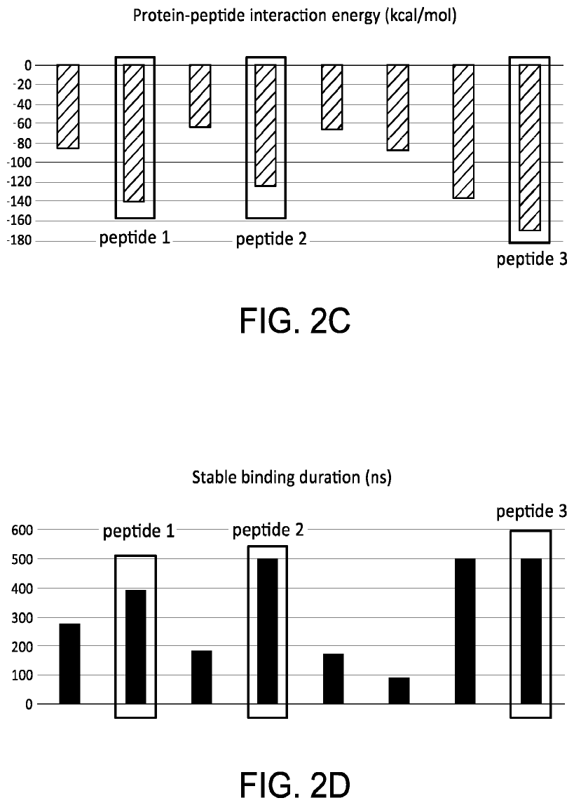

By analyzing the NTD conformations that show agreement with the experimental NMR data, NTD-CTD contacts can be identified, e.g., identifying amino acid residues of the NTD that make contact within an allosteric cavity in the ordered CTD. Targeting this pocket with peptides and/or small molecules could inhibit the binding of the NTD to the CTD. To design the inhibitory peptides, a few backbone templates are initially selected based on the ensemble of NTD conformations that show agreement with NMR. The NTD conformations are clustered by similarity and the representative NTD conformations from the most populated clusters are selected for template design. For each selected NTD conformation, residues on each side of the amino acid residues that locate at the protein-protein interface can be retained as part of the template. The different peptide templates can be considered for the in-silico design. The steps involved in obtaining the peptide templates from a protein NTD ensemble are shown in FIG. 2A.

Starting from a given peptide template, each residue is systematically mutated to all 20 amino acids and an affinity score is calculated using the software Maestro™ (Schrodinger LLC.), which represents the improvement in affinity of the mutant peptide over the starting NTD sequence. The top scoring mutations are analyzed to identify 2-3 positions in each template that were most amenable to mutagenesis. These positions are then mutated combinatorically to generate multiple double and triple mutants, and the top mutants by affinity score are analyzed for features such as strong interaction with the CTD hydrophobic cavity, low desolvation energy and sequence diversity. This step generates peptide candidates, which are then subjected to 500 ns of all atom MD simulations in an explicit water environment, to test their stability of binding to the CTD. Also, the binding free energies are calculated using an MM-GBSA method. Peptides that remain bound within the CTD cavity will show strong interaction with the CTD as measured by the protein-peptide energy and number of hydrogen bonds, and such peptides are selected as peptide candidates for synthesis and further testing. The main steps in selecting the top peptide candidates starting with the NTD templates are described in FIG. 2B.

In embodiments, the disclosure provides method of identifying an amino acid within a disordered domain of a protein that binds to an ordered domain of a protein with the ordered domain either located in the same protein or in a different protein, the method comprising: (i) in silico, performing an enhanced sampling of a disordered domain of a protein binding to an ordered domain of the same protein or an ordered domain of a different protein thereby obtaining an ensemble of conformations, wherein each of the ensemble of conformations comprises the disordered domain bound to the ordered domain; (ii) identifying a first set of structural conformations from the ensemble of conformations that satisfy the experimental structural NMR data of the protein; and (iii) identifying a first amino acid within the first set of structural conformations, wherein the first amino acid is within the disordered domain of the protein that binds to the ordered domain of the same protein or binds to the ordered domain of the different protein. In aspects, the method further comprises (iv) clustering the first set of structural conformations by structural similarity to identify template peptides. In aspects, the methods further comprise identifying a second amino acid within the first set of structural confirmations, wherein the second amino acid is within the ordered domain of the protein that binds to the disordered domain of a protein. In aspects, the first amino acid with the first set of structural conformations comprises at least two amino acids. In aspects, the enhanced sampling comprises accelerated molecular dynamic simulations. In aspects, the enhanced sampling comprises molecular dynamics, Monte Carlo, replica exchange molecular dynamic simulation, metadynamics simulation, temperature cool walking, or generalized simulated annealing. These methods are described graphically in FIG. 2A.

In embodiments, the disclosure provides methods of identifying an amino acid within a disordered domain of a protein that binds to an ordered domain of the same protein or an ordered domain of a different protein, the method comprising: (i) in silico, performing an enhanced sampling of a disordered domain of a protein binding to an ordered domain of a protein thereby obtaining an ensemble of conformations, wherein each of the ensemble of conformations comprises the disordered domain bound to the ordered domain; (ii) identifying a first set of structural conformations from the ensemble of conformations that satisfy the experimental structural NMR data of the protein; and (iii) identifying a first amino acid within the first set of structural conformations, wherein the first amino acid is within the ordered domain of the protein that binds to the disordered domain of the same protein or the disordered domain of a different protein. In aspects, the method further comprises (iv) clustering the first set of structural conformations by structural similarity to identify peptide template peptides. In aspects, the methods further comprise identifying a second amino acid within the first set of structural confirmations, wherein the second amino acid is within the disordered domain of the protein that binds to the ordered domain of a protein. In aspects, the first amino acid with the first set of structural conformations comprises at least two amino acids. In aspects, the enhanced sampling comprises accelerated molecular dynamic simulations. In aspects, the enhanced sampling comprises molecular dynamic simulations, Monte Carlo, replica exchange molecular dynamic simulation, metadynamics simulation, temperature cool walking, or generalized simulated annealing.

In embodiments, the methods further comprise: (a) designing a plurality of template peptides that bind in silico to a first amino acid in the ordered domain based at least in part on the first set of structural conformations; (b) in silico, mutating each residue of each of the plurality of template peptides thereby producing a plurality of mutant peptides; (c) selecting a set of candidate peptides from the plurality of mutant peptides based on in silico binding; (d) synthesizing each of the set of candidate peptides thereby producing a set of synthesized candidate peptides; and (e) experimentally measuring the effect of each of the synthesized candidate peptides to the target protein. In aspects, the term “effect” is binding or any other method that can be used to identify that a peptide is modulating (e.g., inhibiting) the activity of the protein. These methods are described graphically in FIG. 2B.

Computer Systems

In embodiments, the disclosure provides a non-transitory computer readable medium storing instructions, which when executed by at least one data processor, result in operations comprising the methods described herein (e.g., identifying an amino acid within a disordered domain of a protein that binds to an ordered domain of a protein with the ordered domain either located in the same protein or in a different protein, including all embodiments thereof).

In embodiments, the disclosure provides a computer program product comprising a machine-readable medium storing instructions that, when executed by at least one data processor, cause the at least one data processor to perform operations comprising the methods described herein (e.g., identifying an amino acid within a disordered domain of a protein that binds to an ordered domain of a protein with the ordered domain either located in the same protein or in a different protein, including all embodiments thereof).

In embodiments, the disclosure provides a system comprising computer hardware configured to perform operations comprising the methods described herein (e.g., identifying an amino acid within a disordered domain of a protein that binds to an ordered domain of a protein with the ordered domain either located in the same protein or in a different protein, including all embodiments thereof).

In embodiments, the disclosure provides a computer-implemented method comprising the methods described herein (e.g., identifying an amino acid within a disordered domain of a protein that binds to an ordered domain of a protein with the ordered domain either located in the same protein or in a different protein, including all embodiments thereof).

In embodiments, the disclosure provides computer control systems that are programmed to implement the methods described herein (e.g., identifying an amino acid within a disordered domain of a protein that binds to an ordered domain of a protein with the ordered domain either located in the same protein or in a different protein, including all embodiments thereof). A computer system can be programmed or otherwise configured to implements methods of the disclosure, including all embodiments thereof. The computer system can be integral to implementing methods provided herein, which may be otherwise difficult to perform in the absence of the computer system. The computer system can be an electronic device of a user or a computer system that is remotely located with respect to the electronic device. The electronic device can be a mobile electronic device. As an alternative, the computer system can be a computer server.

The computer system includes a central processing unit (CPU, also “processor” and “computer processor”), which can be a single core or multi-core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit (or data repository) for storing data. The computer system can be operatively coupled to a computer network (“network”) with the aid of a communication interface. The network can be the internet, an Internet and/or extranet, or an intranet and/or extranet that is in communication with the internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server. The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback. The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The memory can be part of a database. The machine executable or machine readable code can be provided in the form of software. During use, the code can be executed by the processor. In embodiments, the code can be retrieved from the storage unit and stored on the memory for ready access by the processor. In embodiments, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the internet or other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible storage media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a flooppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, genetic information, such as an identification of disease-causing alleles in single individuals or groups of individuals. Examples of UI's include, without limitation, a graphical user interface (GUI) and web-based user interface (or web interface).

Methods and systems of the present disclosure can be implemented by way of one or more algorithms. An algorithm can be implemented by way of software upon execution by the central processing unit. The algorithm can, for example, prioritize a set of two or more rare genetic variants based on a risk score of each of the two or more rare genetic variants.

In embodiments, the software programs described herein include a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in embodiments, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In embodiments, a web application is written in one or more markup languages, presentation definition languages, client-side scripting languages, server-side coding languages, database query languages, or combinations thereof. In embodiments, a web application is written to some extent in a markup language such as Hypertext Markup Language (HTML), Extensible Hypertext Markup Language (XHTML), or extensible Markup Language (XML). In embodiments, a web application is written to some extent in a presentation definition language such as Cascading Style Sheets (CSS). In embodiments, a web application is written to some extent in a client-side scripting language such as Asynchronous Javascript and XML (AJAX), Flash® Actionscript, Javascript, or Silverlight®. In embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In embodiments, a web application is written to some extent in a database query language such as Structured Query Fanguage (SQF). A web application may integrate enterprise server products such as IBM® Fotus Domino®. A web application may include a media player element. A media player element may utilize one or more of many suitable multimedia technologies including, by way of non limiting examples, Adobe® Flash®, HTMF 5, Apple® QuickTime®, Microsoft® Silverlight®, Java™, and Unity®.

In embodiments, software programs described herein include a mobile application provided to a mobile digital processing device. The mobile application may be provided to a mobile digital processing device at the time it is manufactured. The mobile application may be provided to a mobile digital processing device via the computer network described herein. A mobile application is created by techniques known to those of skill in the art using hardware, languages, and development environments known to the art. Those of skill in the art will recognize that mobile applications may be written in several languages. Suitable programming languages include, by way of non limiting examples, C, C++, C #, Featureive-C, Java™ Javascript, Pascal, Feature Pascal, Python™, Ruby, VB.NET, WMF, and XHTMF/HTMF with or without CSS, or combinations thereof. Suitable mobile application development environments are available from several sources. Commercially available development environments include, by way of non-limiting examples, AirplaySDK, alcheMo, Appcelerator®, Celsius, Bedrock, Flash Fite, .NET Compact Framework, Rhomobile, and WorkFight Mobile Platform. Other development environments may be available without cost including, by way of non-limiting examples, Fazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK. Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In embodiments, the software programs described herein include a standalone application, which is a program that may be run as an independent computer process, not an add-on to an existing process, e.g., not a plug-in. Those of skill in the art will recognize that standalone applications are sometimes compiled. In embodiments, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Perl, R, Python™, Visual Basic, and VB .NET, or combinations thereof. Compilation may be often performed, at least in part, to create an executable program. In embodiments, a computer program includes one or more executable complied applications.

Disclosed herein are software programs that, in embodiments, include a web browser plug-in. In computing, a plug-in, in embodiments, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands. Those skilled in the art will recognize that several plug-in frameworks are available that enable development of plug-ins in various programming languages, including, by way of non-limiting examples, C++, Delphi, Java™, PHP, Python™, and VB .NET, or combinations thereof.

In embodiments, web browsers (also called internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in embodiments, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

The medium, method, and system disclosed herein comprise one or more software, servers, and database modules, or use of the same. In view of the disclosure provided herein, software modules may be created by techniques known to those of skill in the art using machines, software, and languages known to the art. The software modules disclosed herein may be implemented in a multitude of ways. In embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

The medium, method, and system disclosed herein comprise one or more databases. Those of skill in the art will recognize that many databases are suitable for storage and retrieval of information. Suitable databases include, by way of non-limiting examples, relational databases, non-relational databases, feature oriented databases, feature databases, entity-relationship model databases, associative databases, and XML databases. In embodiments, a database is internet-based. In embodiments, a database is web-based. In embodiments, a database is cloud computing-based. A database may be based on one or more local computer storage devices.

The methods, systems, and media described herein, are configured to be performed in one or more facilities at one or more locations. Facility locations are not limited by country and include any country or territory. In embodiments, one or more steps of a method herein are performed in a different country than another step of the method. In embodiments, one or more steps for obtaining a sample are performed in a different country than one or more steps for analyzing a genotype of a sample. In embodiments, one or more method steps involving a computer system are performed in a different country than another step of the methods provided herein. In embodiments, data processing and analyses are performed in a different country or location than one or more steps of the methods described herein. In embodiments, one or more articles, products, or data are transferred from one or more of the facilities to one or more different facilities for analysis or further analysis. An article includes, but is not limited to, one or more components obtained from a sample of a subject and any article or product disclosed herein as an article or product. Data includes, but is not limited to, information regarding genotype and any data produced by the methods disclosed herein. In embodiments of the methods and systems described herein, the analysis is performed and a subsequent data transmission step will convey or transmit the results of the analysis.

In embodiments, any step of any method described herein is performed by a software program or module on a computer. In embodiments, data from any step of any method described herein is transferred to and from facilities located within the same or different countries, including analysis performed in one facility in a particular location and the data shipped to another location or directly to an individual in the same or a different country. In embodiments, data from any step of any method described herein is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

Embodiments disclosed herein provide one or more non-transitory computer readable storage media encoded with a software program including instructions executable by the operating system. In embodiments, software encoded includes one or more software programs described herein. In embodiments, a computer readable storage medium is a tangible component of a computing device. In embodiments, a computer readable storage medium is optionally removable from a computing device. In embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In embodiments, the program and instructions are permanently, substantially permanently, semi-permanently, or non-transitorily encoded on the media.

Galectin-3 Inhibitors

The disclosure provides Galectin-3 inhibitors. In aspects, the Galectin-3 inhibitor is a compound capable of inhibiting an interaction between a disordered N-terminal domain of Galectin-3 and an allosteric cavity in a C-terminal domain of Galectin-3. In aspects, the Galectin-3 inhibitor is a compound that is capable of inhibiting an interaction between the disordered N-terminal domain of Galectin-3 and the allosteric cavity in the C-terminal domain of Galectin-3, wherein the C-terminal domain does not include β-strands β1, β10, β3, β4, β5, and β6. In aspects, the Galectin-3 inhibitor is a compound that is capable of inhibiting an interaction between at least one amino acid in the disordered N-terminal domain of Galectin-3 and at least one amino acid in the allosteric cavity in the C-terminal domain of Galectin-3. In aspects, the Galectin-3 inhibitor is a compound that is capable of inhibiting an interaction between at least one amino acid in the disordered N-terminal domain of Galectin-3 and at least one amino acid in the allosteric cavity in the C-terminal domain of Galectin-3, wherein the C-terminal domain of Galectin-3 does not include β-strands β1, β10, β3, β4, β5, and β6.

In aspects, the Galectin-3 inhibitor is a compound that is capable of inhibiting an interaction between the disordered N-terminal domain of Galectin-3 and the F-face in the C-terminal domain of Galectin-3. In aspects, the Galectin-3 inhibitor is a compound that is capable of inhibiting an interaction between at least one amino acid in the disordered N-terminal domain of Galectin-3 and at least one amino acid in the F-face in a C-terminal domain of Galectin-3. In aspects, the at least one amino acid in the F-face in the C-terminal domain of Galectin-3 is in a β strand selected from the group consisting of β11, β2, β7, β8, β9; a connecting amino acid in the loop between β8 and β9; and a connecting amino acid in the loop between β7 and β8. In aspects, the at least one amino acid in the F-face in the C-terminal domain of Galectin-3 is in a β strand selected from the group consisting of β11, β2, β7, β8, and β9. In aspects, the at least one amino acid in the F-face in the C-terminal domain of Galectin-3 is in a β strand selected from the group consisting of β2, β7, β8, and β9. In aspects, the at least one amino acid in the F-face in the C-terminal domain of Galectin-3 is in a β strand selected from the group consisting of β7, β8, and β9. In aspects, the at least one amino acid in the F-face in the C-terminal domain of Galectin-3 is a connecting amino acid in the loop between β8 and β9, or a connecting amino acid in the loop between β7 and β8. In aspects, the at least one amino acid in the F-face in the C-terminal domain of Galectin-3 is a connecting amino acid in the loop between β8 and β9. In aspects, the connecting amino acid in the loop between β8 and β9 is selected from the group consisting of N214 and D215. In aspects, the at least one amino acid in the F-face in the C-terminal domain of Galectin-3 is a connecting amino acid in the loop between β7 and β8. In aspects, the connecting amino acid in the loop between β8 and β7 is selected from the group consisting of E205, P206, and D207. In aspects, the connecting amino acid in the loop between β8 and β7 is E205. In aspects, the at least one amino acid is in the β11 strand on the F-face in the C-terminal domain of Galectin-3. In aspects, the at least one amino acid is in the β2 strand on the F-face in the C-terminal domain of Galectin-3. In aspects, the at least one amino acid in the β2 strand on the F-face in the C-terminal domain of Galectin-3 is selected from the group consisting of L131 and I132. In aspects, the at least one amino acid in the β2 strand on the F-face in the C-terminal domain of Galectin-3 is L131. In aspects, the at least one amino acid in the β2 strand on the F-face in the C-terminal domain of Galectin-3 is I132. In aspects, the at least one amino acid is in the β7 strand on the F-face in the C-terminal domain of Galectin-3. In aspects, the at least one amino acid in the β7 strand on the F-face in the C-terminal domain of Galectin-3 is selected from the group consisting of K199, Q201, V202, L203, and V204. In aspects, the at least one amino acid in the β7 strand on the F-face in the C-terminal domain of Galectin-3 is selected from the group consisting of V202, L203, and V204. In aspects, the at least one amino acid in the β7 strand on the F-face in the C-terminal domain of Galectin-3 is selected from the group consisting of V202 and V204. In aspects, the at least one amino acid in the β7 strand on the F-face in the C-terminal domain of Galectin-3 is V202. In aspects, the at least one amino acid in the β7 strand on the F-face in the C-terminal domain of Galectin-3 is L203. In aspects, the at least one amino acid in the β7 strand on the F-face in the C-terminal domain of Galectin-3 is V204. In aspects, the at least one amino acid is in the β8 strand on the F-face in the C-terminal domain of Galectin-3. In aspects, the at least one amino acid in the β8 strand on the F-face in the C-terminal domain of Galectin-3 is selected from the group consisting of P209, H208, K210, V211, A212, and V213. In aspects, the at least one amino acid in the β8 strand on the F-face in the C-terminal domain of Galectin-3 is selected from the group consisting of K210, V211, A212, and V213. In aspects, the at least one amino acid in the β8 strand on the F-face in the C-terminal domain of Galectin-3 is K210. In aspects, the at least one amino acid in the β8 strand on the F-face in the C-terminal domain of Galectin-3 is V211. In aspects, the at least one amino acid in the β8 strand on the F-face in the C-terminal domain of Galectin-3 is A212. In aspects, the at least one amino acid in the β8 strand on the F-face in the C-terminal domain of Galectin-3 is V213. In aspects, the at least one amino acid is in the β9 strand on the F-face in the C-terminal domain of Galectin-3. In aspects, the at least one amino acid in the β9 strand on the F-face in the C-terminal domain of Galectin-3 is selected from the group consisting of A216, H217, L218, and L219. In aspects, the at least one amino acid in the β9 strand on the F-face in the C-terminal domain of Galectin-3 is selected from the group consisting of A216, L218, and L219. In aspects, the at least one amino acid in the β9 strand on the F-face in the C-terminal domain of Galectin-3 is A216. In aspects, the at least one amino acid in the β9 strand on the F-face in the C-terminal domain of Galectin-3 is L218. In aspects, the at least one amino acid in the β9 strand on the F-face in the C-terminal domain of Galectin-3 is L219.

In aspects, the Galectin-3 inhibitor is a compound that is capable of inhibiting an interaction between at least one amino acid in the disordered N-terminal domain of Galectin-3 and at least one amino acid in the C-terminal domain of Galectin-3; wherein the at least one amino acid in the C-terminal domain of Galectin-3 is in: (i) a β strand selected from the group consisting of β11, β2, β7, β8, and β9; (ii) an amino acid in the loop between β8 and (39; (iii) an amino acid in the loop between β7 and β8; (iv) an amino acid in the loop between β2 and β3; or (v) an amino acid in the loop between β1 and β2. In aspects, the Galectin-3 inhibitor is a compound that is capable of inhibiting an interaction between at least one amino acid in the disordered N-terminal domain of Galectin-3 and at least one amino acid in the C-terminal domain of Galectin-3; wherein the at least one amino acid in the C-terminal domain of Galectin-3 is in: (i) an amino acid in the loop between β8 and β9; (ii) an amino acid in the loop between β7 and β8; (iii) an amino acid in the loop between β2 and β3; or (iv) an amino acid in the loop between β1 and β2. In aspects, the Galectin-3 inhibitor is a compound that is capable of inhibiting an interaction between at least one amino acid in the disordered N-terminal domain of Galectin-3 and at least one amino acid in the C-terminal domain of Galectin-3; wherein the at least one amino acid in the C-terminal domain of Galectin-3 is in: (i) an amino acid in the loop between β2 and β3 or (ii) an amino acid in the loop between β1 and β2. In aspects, the Galectin-3 inhibitor is a compound that is capable of inhibiting an interaction between at least one amino acid in the disordered N-terminal domain of Galectin-3 and at least one amino acid in the C-terminal domain of Galectin-3; wherein the at least one amino acid in the C-terminal domain of Galectin-3 is in an amino acid in the loop between β2 and β3. In aspects, the Galectin-3 inhibitor is a compound that is capable of inhibiting an interaction between at least one amino acid in the disordered N-terminal domain of Galectin-3 and at least one amino acid in the C-terminal domain of Galectin-3; wherein the at least one amino acid in the C-terminal domain of Galectin-3 is in an amino acid in the loop between β1 and β2. In aspects, the at least one amino acid in the disordered N-terminal domain of Galectin-3 is selected from the group consisting of A2, A49, A53, A69, D3, F5, G108, G112, G43, G47, G52, G68, G72, H8, P106, P71, Q20, Q48, S84, T98, V78, W22, Y101, Y41, Y36, Y45, Y54, Y70, Y79, T104, Y89, and A100.

In aspects, the Galectin-3 inhibitor is a compound that is capable of inhibiting an interaction between at least one amino acid in the disordered N-terminal domain of Galectin-3 and at least one amino acid selected from the group consisting of Y247, T243, Q201, V202, K210, A216, F192, F198, K199, L203, V204, D215, H217, Q220, L219, L131, V211, A212, V213, L218, E205, and I132 in the allosteric cavity in the C-terminal domain of Galectin-3. In aspects, the Galectin-3 inhibitor is a compound that is capable of inhibiting an interaction between at least one amino acid selected from the group consisting of A2, A49, A53, A69, D3, F5, G108, G112, G43, G47, G52, G68, G72, H8, P106, P71, Q20, Q48, S84, T98, V78, W22, Y101, Y41, Y36, Y45, Y54, Y70, Y79, T104, Y89, and A100 in the disordered N-terminal domain of Galectin-3 and at least one amino acid in the allosteric cavity in the C-terminal domain of Galectin-3. In aspects, the Galectin-3 inhibitor is a compound that is capable of inhibiting an interaction between at least one amino acid selected from the group consisting of A2, A49, A53, A69, D3, F5, G108, G112, G43, G47, G52, G68, G72, H8, P106, P71, Q20, Q48, S84, T98, V78, W22, Y101, Y41, Y36, Y45, Y54, Y70, Y79, T104, Y89, and A100 in the disordered N-terminal domain of Galectin-3 and at least one amino acid selected from the group consisting of Y247, T243, Q201, V202, K210, A216, F192, F198, K199, L203, V204, D215, H217, Q220, L219, L131, V211, A212, V213, L218, E205, and I132 in the allosteric cavity in the C-terminal domain of Galectin-3. In aspects, the allosteric cavity in the C-terminal domain of Galectin-3 is the F-face in the C-terminal domain of Galectin-3.

In aspects, the amino acid in the disordered N-terminal domain is Y36 and/or Y45. In aspects, the amino acid in the disordered N-terminal domain is Y36. In aspects, the amino acid in the disordered N-terminal domain is Y45. In aspects, the amino acid in the disordered N-terminal domain of Galectin-3 is selected from the group consisting of A2, A49, A53, A69, D3, F5, G108, G112, G43, G47, G52, G68, G72, H8, P106, P71, Q20, Q48, S84, T98, V78, W22, Y101, Y41, Y36, Y45, Y54, Y70, and Y79. In aspects, the amino acid in the disordered N-terminal domain of Galectin-3 is selected from the group consisting of W22, Y101, Y41, Y36, Y45, Y54, Y70, G47, Q48, A73, Y79, T98, P71, T104, P106, Y89, A100, and G112. In aspects, the amino acid in the disordered N-terminal domain of Galectin-3 is selected from the group consisting of Y247, T243, Q201, V202, K210, A216, F192, F198, K199, L203, V204, D215, H217, Q220, L219, L131, V211, A212, V213, L218, E205, and I132. In aspects, the amino acid in the disordered N-terminal domain of Galectin-3 is selected from the group consisting of Y41, Y45, G47, and Q48. In aspects, the amino acid in the disordered N-terminal domain of Galectin-3 is selected from the group consisting of Y79, A73, T104, T98, P71, P106, Y89, and Y54. In aspects, the allosteric cavity in the C-terminal domain of Galectin-3 is the F-face in the C-terminal domain of Galectin-3.

In aspects, the amino acid in the C-terminal domain of Galectin-3 is selected from the group consisting of L131, L203, H217, Q201, and D215. In aspects, the amino acid in the C-terminal domain of Galectin-3 is selected from the group consisting of L131, L203, and H217. In aspects, the amino acid in the C-terminal domain of Galectin-3 is selected from the group consisting of K210, V211, A212, V213, A216, L218, L219, V202, V204, and E205. In aspects, the amino acid in the C-terminal domain of Galectin-3 is selected from the group consisting of K210, V211, A212, V213, A216, L218, and L219. In aspects, the amino acid in the C-terminal domain of Galectin-3 is selected from the group consisting of K210, V211, A212, and V213. In aspects, the amino acid in the C-terminal domain of Galectin-3 is selected from the group consisting of A216, L218, L219. In aspects, the amino acid in the C-terminal domain of Galectin-3 is selected from the group consisting of A216, L218, L219, V213, A212, V211, K210, V202, V204, 1132. In aspects, the allosteric cavity in the C-terminal domain of Galectin-3 is the F-face in the C-terminal domain of Galectin-3.

In aspects, the phrase “the amino acid . . . is selected from the group consisting of” means that at least one amino is selected from the group. In aspects, the phrase “the amino acid . . . is selected from the group consisting of” means that at least two amino acids are selected from the group. In aspects, the phrase “the amino acid . . . is selected from the group consisting of” means that at least three amino acids are selected from the group. In aspects, the phrase “the amino acid . . . is selected from the group consisting of” means that at least four amino acids are selected from the group. In aspects, the phrase “the amino acid . . . is selected from the group consisting of” means that at least five amino acids are selected from the group. In aspects, the phrase “the amino acid . . . is selected from the group consisting of” means that one amino acid is selected from the group. In aspects, the phrase “the amino acid . . . is selected from the group consisting of” means that two amino acids are selected from the group. In aspects, the phrase “the amino acid . . . is selected from the group consisting of” means that three amino acids are selected from the group. In aspects, the phrase “the amino acid . . . is selected from the group consisting of” means that four amino acids are selected from the group. In aspects, the phrase “the amino acid . . . is selected from the group consisting of” means that five amino acids are selected from the group.

In aspects, the Galectin-3 inhibitor is a compound that is capable of inhibiting an interaction between Y36 and/or Y45 in the disordered N-terminal domain of Galectin-3 and V202, K210, A216, F192, F198, K199, L203, V204, D215, H217, Q220, L219, or a combination of two or more thereof in the allosteric cavity in the C-terminal domain of Galectin-3. In aspects, the Galectin-3 inhibitor is a compound that is capable of inhibiting an interaction between Y36 in the disordered N-terminal domain of Galectin-3 and V202, K210, A216, F192, F198, K199, L203, V204, D215, H217, Q220, L219, or a combination of two or more thereof in the allosteric cavity in the C-terminal domain of Galectin-3. In aspects, the Galectin-3 inhibitor is a compound that is capable of inhibiting an interaction between Y45 in the disordered N-terminal domain of Galectin-3 and V202, K210, A216, F192, F198, K199, L203, V204, D215, H217, Q220, L219, or a combination of two or more thereof in the allosteric cavity in the C-terminal domain of Galectin-3. In aspects, the allosteric cavity in the C-terminal domain of Galectin-3 is the F-face in the C-terminal domain of Galectin-3.

In aspects, the Galectin-3 inhibitor is a compound that is capable of inhibiting an interaction between Y36 and/or Y45 in the disordered N-terminal domain of Galectin-3 and V202, K210, A216, or a combination of two or more thereof in the allosteric cavity in the C-terminal domain of Galectin-3. In aspects, the Galectin-3 inhibitor is a compound that is capable of inhibiting an interaction between Y36 in the disordered N-terminal domain of Galectin-3 and V202, K210, A216, or a combination of two or more thereof in the allosteric cavity in the C-terminal domain of Galectin-3. In aspects, the Galectin-3 inhibitor is a compound that is capable of inhibiting an interaction between Y45 in the disordered N-terminal domain of Galectin-3 and V202, K210, A216, or a combination of two or more thereof in the allosteric cavity in the C-terminal domain of Galectin-3. In aspects, the allosteric cavity in the C-terminal domain of Galectin-3 is the F-face in the C-terminal domain of Galectin-3.

In embodiments, the Galectin-3 inhibitor is a peptide. In aspects, the Galectin-3 inhibitor is a small molecule. In aspects, “small molecule” is a low molecular weight (e.g., 1,000 Daltons or less) organic compound. In aspects, the Galectin-3 inhibitor is a macrocycle. In aspects, a macrocycle is a molecule and/or ion containing twelve or more membered ring. In aspects, the Galectin-3 inhibitor has an inhibitor effect on Galectin-3 that is the same as or better than the peptide comprising the amino acid sequence of SEQ ID NO:9 and/or that fills the same space as the peptide comprising the amino acid sequence of SEQ ID NO:9. In aspects, the term “that is the same as” means +/−10%. In aspects, the Galectin-3 inhibitor is covalently bonded to: (i) a delivery agent, (ii) a detectable agent, or (iii) a delivery agent and a detectable agent. In aspects, the Galectin-3 inhibitor is covalently bonded to a delivery agent. In aspects, the Galectin-3 inhibitor is covalently bonded to a detectable agent. In aspects, the Galectin-3 inhibitor is covalently bonded to a delivery agent and a detectable agent.

In embodiments, the Galectin-3 inhibitor comprises the peptide of any one of SEQ ID NOS:1-9. In embodiments, the Galectin-3 inhibitor comprises the peptide of any one of SEQ ID NOS:1-9. In aspects, the Galectin-3 inhibitor comprises the peptide of SEQ ID NO:3. In aspects, the Galectin-3 inhibitor comprises the peptide of SEQ ID NO:9. In aspects, one or more of the amino acid residues in SEQ ID NO:3 is phosphorylated, nitrogen methylated, or sulfated. In aspects, one or more of the tyrosine residues in SEQ ID NO:3 is phosphorylated, nitrogen methylated, or sulfated. In aspects, one or more of the amino acid residues in SEQ ID NO:9 is phosphorylated, nitrogen methylated, or sulfated. In aspects, one or more of the tyrosine residues in SEQ ID NO:9 is phosphorylated, nitrogen methylated, or sulfated.

In embodiments, the disclosure provides a peptide comprising SEQ ID NO:3, also referred to herein as Peptide 3. In aspects, the disclosure provides peptides comprising amino acid sequence having at least 90% sequence identity to the amino acid sequence of SEQ ID NO:3. In aspects, the disclosure provides peptides comprising amino acid sequence having at least 95% sequence identity to the amino acid sequence of SEQ ID NO:3. In aspects, the disclosure provides peptides comprising an amino acid sequence that differs by 1-3 amino acids from the amino acid sequence of SEQ ID NO:3. In aspects, the disclosure provides peptides comprising an amino acid sequence that differs by 2 amino acids from the amino acid sequence of SEQ ID NO:3. In aspects, the disclosure provides peptides comprising an amino acid sequence that differs by 1 amino acid from the amino acid sequence of SEQ ID NO:3. In aspects, the disclosure provides peptides having from 1 to 5 additional amino acids on the N-terminus and/or on the C-terminus of the peptide comprising the amino acid sequence of SEQ ID NO:3. In aspects, the N-terminus is an amide or the N-terminus is a capped-amide. In aspects, the N-terminus is an acetyl-capped amide. In aspects, the C-terminus is a carboxyl group or the C-terminus is a capped-carboxyl group. In aspects, the C-terminus is an amide-capped carboxyl group. In aspects, the N-terminus is an acetyl-capped amide and the C-terminus is an amide-capped carboxyl group. In embodiments, the disclosure provides an isolated nucleic acid that encodes the amino acid sequence of SEQ ID NO:3, including embodiments and aspects thereof, as described herein. In embodiments, the disclosure provides a vector (e.g., plasmid, viral vector) which comprises a nucleic acid that encodes the amino acid sequence of SEQ ID NO:3, including embodiments and aspects thereof, as described herein.

In embodiments, the disclosure provides a peptide comprising SEQ ID NO:9. In aspects, the disclosure provides peptides comprising amino acid sequence having at least 90% sequence identity to the amino acid sequence of SEQ ID NO:9. In aspects, the disclosure provides peptides comprising amino acid sequence having at least 95% sequence identity to the amino acid sequence of SEQ ID NO:9. In aspects, the disclosure provides peptides having from 1 to 25 additional amino acids on the N-terminus and/or on the C-terminus of the peptide comprising the amino acid sequence of SEQ ID NO:9. In aspects, the disclosure provides peptides having from 1 to 20 additional amino acids on the N-terminus and/or on the C-terminus of the peptide comprising the amino acid sequence of SEQ ID NO:9. In aspects, the disclosure provides peptides having from 1 to 15 additional amino acids on the N-terminus and/or on the C-terminus of the peptide comprising the amino acid sequence of SEQ ID NO:9. In aspects, the disclosure provides peptides having from 1 to 10 additional amino acids on the N-terminus and/or on the C-terminus of the peptide comprising the amino acid sequence of SEQ ID NO:9. In aspects, the disclosure provides peptides having from 1 to 5 additional amino acids on the N-terminus and/or on the C-terminus of the peptide comprising the amino acid sequence of SEQ ID NO:9. In aspects, the N-terminus is an amide or the N-terminus is a capped-amide. In aspects, the N-terminus is an acetyl-capped amide. In aspects, the C-terminus is a carboxyl group or the C-terminus is a capped-carboxyl group. In aspects, the C-terminus is an amide-capped carboxyl group. In aspects, the N-terminus is an acetyl-capped amide and the C-terminus is an amide-capped carboxyl group. In embodiments, the disclosure provides an isolated nucleic acid that encodes the amino acid sequence of SEQ ID NO:9, including embodiments and aspects thereof, as described herein. In embodiments, the disclosure provides a vector (e.g., plasmid, viral vector) which comprises a nucleic acid that encodes the amino acid sequence of SEQ ID NO:9, including embodiments and aspects thereof, as described herein.

In embodiments, the disclosure provides peptides comprising any one of SEQ ID NOS:1-9. In aspects, any one of SEQ ID NOS:1-9 have an N-terminus capped with an acetyl group. In aspects, any one of SEQ ID NOS:1-9 has a C-terminus capped with an amide group. In aspects, the disclosure provides a peptide comprising SEQ ID NO:1. In aspects, the disclosure provides a peptide comprising SEQ ID NO:2. In aspects, the disclosure provides a peptide comprising SEQ ID NO:3. In aspects, the disclosure provides a peptide comprising SEQ ID NO:4. In aspects, the disclosure provides a peptide comprising SEQ ID NO:5. In aspects, the disclosure provides a peptide comprising SEQ ID NO:6. In aspects, the disclosure provides a peptide comprising SEQ ID NO:7. In aspects, the disclosure provides a peptide comprising SEQ ID NO:8. In aspects, the disclosure provides a peptide comprising SEQ ID NO:9. In aspects, the disclosure provides peptides comprising amino acid sequence having at least 90% sequence identity to the amino acid sequence of any one of SEQ ID NOS:1-9. In aspects, the disclosure provides peptides comprising amino acid sequence having at least 95% sequence identity to the amino acid sequence of any one of SEQ ID NOS:1-9. In aspects, the disclosure provides peptides comprising amino acid sequence that differs by 1-3 amino acids from the amino acid sequence of any one of SEQ ID NOS:1-9. In aspects, the disclosure provides peptides comprising amino acid sequence that differs by 2 amino acids from the amino acid sequence of any one of SEQ ID NOS:1-9. In aspects, the disclosure provides peptides comprising amino acid sequence that differs by 1 amino acid from the amino acid sequence of any one of SEQ ID NOS:1-9. In aspects, the disclosure provides peptides having from 1 to 5 additional amino acids on the N-terminus and/or on the C-terminus of the peptide comprising the amino acid sequence of any one of SEQ ID NOS:1-9. In aspects, the N-terminus is an amide or the N-terminus is a capped-amide. In aspects, the N-terminus is an acetyl-capped amide. In aspects, the C-terminus is a carboxyl group or the C-terminus is a capped-carboxyl group. In aspects, the C-terminus is an amide-capped carboxyl group. In aspects, the N-terminus is an acetyl-capped amide and the C-terminus is an amide-capped carboxyl group. In aspects, one or more of the amino acid residues in any one of SEQ ID NOS:1-9 is phosphorylated, nitrogen methylated, or sulfated. In aspects, one or more of the tyrosine residues in any one of SEQ ID NOS:1-9 is phosphorylated, nitrogen methylated, or sulfated. In embodiments, the disclosure provides an isolated nucleic acid that encodes the amino acid sequence of any one of SEQ ID NOS:1-9, including embodiments and aspects thereof, as described herein. In embodiments, the disclosure provides a vector (e.g., plasmid, viral vector) which comprises a nucleic acid that encodes the amino acid sequence of any one of SEQ ID NOS:1-9, including embodiments and aspects thereof, as described herein.

In embodiments, the disclosure provides a compound comprising the peptide of SEQ ID NO:3 covalently bonded to: (i) a peptide delivery agent, (ii) a detectable agent, or (iii) a peptide delivery agent and a detectable agent. In aspects, the compound comprises the peptide of SEQ ID NO:3 covalently bonded to a peptide delivery agent. In aspects, the compound comprises the peptide of SEQ ID NO:3 covalently bonded to a detectable agent. In aspects, the compound comprises the peptide of SEQ ID NO:3 covalently bonded to a peptide delivery agent and a detectable agent. The peptide of SEQ ID NO:3 can be in the form of any of the embodiments and aspects described herein.

In embodiments, the disclosure provides a compound comprising the peptide of SEQ ID NO:9 covalently bonded to: (i) a peptide delivery agent, (ii) a detectable agent, or (iii) a peptide delivery agent and a detectable agent. In aspects, the compound comprises the peptide of SEQ ID NO:9 covalently bonded to a peptide delivery agent. In aspects, the compound comprises the peptide of SEQ ID NO:9 covalently bonded to a detectable agent. In aspects, the compound comprises the peptide of SEQ ID NO:9 covalently bonded to a peptide delivery agent and a detectable agent. The peptide of SEQ ID NO:9 can be in the form of any of the embodiments and aspects described herein.

In embodiments, the disclosure provides a compound comprising the peptide of any one of SEQ ID NOS:1-9 covalently bonded to: (i) a peptide delivery agent, (ii) a detectable agent, or (iii) a peptide delivery agent and a detectable agent. In aspects, the compound comprises the peptide of any one of SEQ ID NOS:1-9 covalently bonded to a peptide delivery agent. In aspects, the compound comprises the peptide of any one of SEQ ID NOS:1-9 covalently bonded to a detectable agent. In aspects, the compound comprises the peptide of any one of SEQ ID NOS:1-9 covalently bonded to a peptide delivery agent and a detectable agent.

In aspects, the peptide of any one of SEQ ID NOS:1-9 is covalently bonded via a linking group to (i) a peptide delivery agent, (ii) a detectable agent, or (iii) a peptide delivery agent and a detectable agent. The linking group can be any known in the art. In aspects, the linking group comprises amino acids, DNA (both single and double stranded), RNA, a chemical linking group, or a combination thereof. In aspects, the linking group comprises amino acids (e.g., 1 to about 20 amino acids). In aspects, the chemical linking group comprises substituted or unsubstituted alkylene, substituted or unsubstituted heteroalkylene, substituted or unsubstituted arylene, substituted or unsubstituted heteroarylene, substituted or unsubstituted cycloalkylene, substituted or unsubstituted heterocycloalkylene, or a combination of two or more thereof.

A “detectable agent” is a compound or composition detectable by appropriate means such as spectroscopic, photochemical, biochemical, immunochemical, chemical, magnetic resonance imaging, or other physical means. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In aspects, the detectable agent is a detectable fluorescent agent. In aspects, the detectable agent is a detectable phosphorescent agent. In aspects, the detectable agent is a detectable radioactive agent. In aspects, the detectable agent is a detectable luminescent agent.

“Fluorophore” refers to compounds that absorb light energy of a specific wavelength and re-emit the light at a lower wavelength. Exemplary fluorophores that may be used herein include xanthenes (e.g., fluorescein, rhodamine, Oregon green, eosin, Texas red); cyanines (e.g., cyanine, indocarbocyanine, oxacarbocyanine, thiacarbocyanine, merocyanine); squaraines (e.g., Seta, Square dyes); squaraine rotaxane (e.g., SeTau® dyes); naphthalenes (e.g., dansyl, prodan); coumarins; oxadiazoles (e.g., pyridyloxazole, nitrobenzoxadiazole, benzooxadiazole); anthracenes (e.g., anthraquinones, DRAQ5®, DRAQ7®, CyTRAK® orange); pyrenes (e.g., cascade blue); oxazines (e.g., Nile red, Nile blue, cresyl violet, oxazine 170); acridines (e.g., proflavin, acridine orange, acridine yellow); arylmethines (e.g., auramine, crystal violet, malachite green); tetrapyrroles (e.g., porphin, phthalocyanine, bilirubin); and the like.

Radioactive agents (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴¹r, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The terms “delivery agent” and “peptide delivery agent” refer to any compound or moiety that can deliver in vivo a compound or peptide described herein into a cell of interest and/or to the vicinity of a cell of interest. Cells of interest include cancer cells. In aspects, the delivery agent is a polymer or copolymer. In aspects, the copolymer comprises acrylamide. In aspects, the copolymer is a N-(2-hydroxypropyl) methacrylamide copolymer. Exemplary delivery agents are described, e.g., by Sun et al, Acta Pharmacologica Sinica, 38:806-822 (2017); and Sun et al, Mol Pharm 12(11):4124-4136 (2015).

Compositions

Provided herein are pharmaceutical compositions comprising an active ingredient (e.g., a Galectin-3 inhibitor) and a pharmaceutically acceptable excipient. The term “active ingredient” refers to Galectin-3 inhibitors (including Galectin-3 inhibitors covalently bonded to delivery agents and/or detectable agents). In embodiments, the active ingredient is a peptide comprising SEQ NO:3 as described herein. In embodiments, the active ingredient is a peptide comprising SEQ NO:9 as described herein. The compositions are suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. Pharmaceutical compositions can be used for treating a disease and/or for detecting (e.g., imaging) a disease without treating the disease.

Compositions can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations of the compositions may be administered depending on the dosage and frequency as required and tolerated by the patient.

Pharmaceutical compositions provided herein include compositions wherein the active ingredient (e.g., a Galectin-3 inhibitor described herein, including embodiments or aspects thereof) is contained in an effective amount, i.e., in an amount effective to achieve its intended purpose. The actual amount effective for a particular application will depend, inter alia, on the condition being treated. When administered in methods to treat a disease, the compounds described herein will contain an amount of active ingredient effective to achieve the desired result, e.g., modulating the activity of a target molecule, and/or reducing, eliminating, or slowing the progression of a disease or symptoms thereof. Determination of a therapeutically effective amount of a compound described herein is well within the capabilities of the skilled artisan, especially in light of the detailed disclosure herein.

The pharmaceutical compositions can include a single active ingredient or more than one active ingredient. The compositions for administration will commonly include an active ingredient as described herein dissolved, dispersed, or suspended in a pharmaceutically acceptable carrier, such as an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable excipients as required to approximate physiological conditions such as pH adjusting and buffering agents, toxicity adjusting agents and the like, for example, sodium acetate, sodium chloride, potassium chloride, calcium chloride, sodium lactate and the like. The concentration of active ingredient in these formulations can vary, and will be selected primarily based on fluid volumes, viscosities, body weight and the like in accordance with the particular mode of administration selected and the subject's needs.

Solutions of the active ingredients as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these compositions can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic compositions and appropriate drug stabilizers, if required, may be included in the formulation. Various commercial nasal compositions are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with food. For oral administration, the active ingredients may be incorporated with excipients and used in the form of ingestible tablets, buccal tablets, troches, capsules, elixirs, suspensions, syrups, wafers, and the like. Such compositions should contain at least 0.1% of active ingredient. The percentage of the compositions may, of course, be varied and may conveniently be between about 1 to about 90% of the weight of the unit, or preferably between 1-60%. The amount of active ingredient in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active ingredient in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

The compositions can be presented in unit-dose or multi-dose sealed containers, such as ampules and vials. Thus, the composition can be in unit dosage form. In such form the composition is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

“Pharmaceutically acceptable excipient” and “pharmaceutically acceptable carrier” refer to a substance that aids the administration of an active agent to and absorption by a subject and can be included in the compositions herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such compositions can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful.

Methods

The disclosure provides methods for treating diseases characterized by an overexpression of Galectin-3 in a subject in need thereof by administering to the subject an effective amount of the peptides, compounds, or compositions described therein (including all embodiments and aspects thereof). Diseases characterized by an overexpression or inappropriate expression of Galectin-3 are known in the art. In aspects, the disease characterized by an overexpression of Galectin-3 is cancer, fibrosis, a cardiovascular disease, an infectious disease, an inflammatory disease, or a neurological disease. Thus, the disclosure provides methods for treating cancer, fibrosis, a cardiovascular disease, an infectious disease, an inflammatory disease, or a neurological disease in a subject in need thereof by administering to the subject an effective amount of the peptides, compounds, or compositions described therein (including all embodiments and aspects thereof).

The disclosure provides methods for treating cancer in a subject in need thereof by administering to the subject an effective amount of the peptides, compounds, or compositions described therein (including all embodiments and aspects thereof). In aspects, the cancer is characterized by overexpression or inappropriate expression of Galectin-3. In aspects, the cancer is leukemia, ovarian cancer, breast cancer, bladder cancer, gastric cancer, prostate cancer, lung cancer, pancreatic cancer, thyroid cancer, colon cancer, melanoma, or lymphoma. In aspects, the cancer is leukemia. In aspects, the cancer is acute lymphoblastic leukemia. In aspects, the cancer is ovarian cancer. In aspects, the cancer is breast cancer. In aspects, the cancer is bladder cancer. In aspects, the cancer is gastric cancer. In aspects, the cancer is prostate cancer. In aspects, the cancer is lung cancer. In aspects, the cancer is pancreatic cancer. In aspects, the cancer is thyroid cancer. In aspects, the cancer is colon cancer. In aspects, the cancer is melanoma. In aspects, the cancer is lymphoma. In aspects, the lung cancer is non-small cell lung cancer. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for detecting cancer in a subject in need thereof by administering to the subject an effective amount of the peptides, compounds, or compositions described therein (including all embodiments and aspects thereof). In aspects, the methods for detecting cancer comprise administering an effective amount of a peptide described herein covalently bonded to a detectable agent. The peptide binds to the overexpressed or inappropriately expressed Galectin-3 in a cancer, such as a solid tumor, and that the detectable agent can be identified through an imaging technique, thereby identifying the presence of a cancer that overexpresses Galectin-3. In aspects, the methods for detecting cancer comprise administering an effective amount of a peptide described herein covalently bonded to a detectable agent and a peptide delivery agent. The peptide binds to the overexpressed or inappropriately expressed Galectin-3 in a cancer, such as a solid tumor, and that the detectable agent can be identified through an imaging technique, thereby identifying the presence of a cancer that overexpresses or inappropriately expresses Galectin-3. If cancer is detected, then the subject can be administered an effective amount of the peptide, compound, or composition (including embodiments and aspects thereof) to treat the cancer. Imaging techniques are known in the art and include, e.g., X-rays, computed tomography (CT) scans, magnetic resonance imaging (MRI), ultrasound, nuclear medicine imagining (e.g., positron-emission tomography (PET)), and the like. In aspects, the cancer is characterized by an overexpression or inappropriate expression of Galectin-3. In aspects, the cancer is leukemia, ovarian cancer, breast cancer, bladder cancer, gastric cancer, prostate cancer, lung cancer, pancreatic cancer, thyroid cancer, melanoma, or lymphoma. In aspects, the cancer is leukemia. In aspects, the cancer is acute lymphoblastic leukemia. In aspects, the cancer is ovarian cancer. In aspects, the cancer is breast cancer. In aspects, the cancer is bladder cancer. In aspects, the cancer is gastric cancer. In aspects, the cancer is prostate cancer. In aspects, the cancer is lung cancer. In aspects, the cancer is pancreatic cancer. In aspects, the cancer is thyroid cancer. In aspects, the cancer is melanoma. In aspects, the cancer is lymphoma. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for treating fibrosis in a subject in need thereof by administering to the subject an effective amount of the peptides, compounds, or compositions described therein (including all embodiments and aspects thereof). In aspects, the fibrosis is cardiac fibrosis, pulmonary fibrosis, liver fibrosis, or kidney fibrosis. In aspects, the fibrosis is pulmonary fibrosis. In aspects, the fibrosis is idiopathic pulmonary fibrosis. In aspects, the fibrosis is liver fibrosis. In aspects, the fibrosis is nonalcoholic steatohepatitis. In aspects, the fibrosis is kidney fibrosis. In aspects, the fibrosis is cardiac fibrosis. In aspects, the fibrosis is tissue fibrosis. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for a cardiovascular disease in a subject in need thereof by administering to the subject an effective amount of the peptides, compounds, or compositions described therein (including all embodiments and aspects thereof). In aspects, the cardiovascular disease is heart failure. In aspects, the cardiovascular disease is atherosclerosis. In aspects, the cardiovascular disease is a cardiovascular disease as described herein. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for treating an infectious disease in a subject in need thereof by administering to the subject an effective amount of the peptides, compounds, or compositions described therein (including all embodiments and aspects thereof). In aspects, the infectious disease is meningitis. In aspects, the infectious disease is a coronavirus infection (e.g., SARS-CoV-1, SARS-CoV-2, MERS-CoV). In aspects, the infectious disease is COVID-19. In aspects, the infectious disease is MERS. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for treating an inflammatory disease in a subject in need thereof by administering to the subject an effective amount of the peptides, compounds, or compositions described therein (including all embodiments and aspects thereof). In aspects, the inflammatory disease is type 1 diabetes. In aspects, the inflammatory disease is type 2 diabetes. In aspects, the inflammatory disease is sepsis. In aspects, the inflammatory disease is acute respiratory distress syndrome. In aspects, the inflammatory disease is caused by degradation of retinal ganglion cells, which can lead to optic nerve injury, retinal ischemia, or glaucoma. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for treating a neurological disease in a subject in need thereof by administering to the subject an effective amount of the peptides, compounds, or compositions described therein (including all embodiments and aspects thereof). In aspects, the neurological disease is Alzheimer's disease. In aspects, the methods comprise administering an effective amount of a peptide comprising SEQ ID NO:3. In aspects, the methods comprise administering an effective amount of a pharmaceutical composition comprising a peptide which comprises SEQ ID NO:3 and a pharmaceutically acceptable excipient. In aspects, the methods comprise administering an effective amount of a peptide comprising SEQ ID NO:9. In aspects, the methods comprise administering an effective amount of a pharmaceutical composition comprising a peptide which comprises SEQ ID NO:9 and a pharmaceutically acceptable excipient.

The disclosure provides methods for treating a disease characterized by overexpression or inappropriate expression of Galectin-3 in a subject in need thereof, the method comprising administering to the subject an effective amount of a compound; wherein the compound that is capable of inhibiting an interaction between a disordered N-terminal domain of Galectin-3 and an allosteric cavity in an ordered C-terminal domain of Galectin-3. In aspects, the disordered N-terminal domain of Galectin-3 comprises from 1 to about 80 contiguous amino acid residues. In aspects, the disordered N-terminal domain of Galectin-3 comprises from 1 to about 60 contiguous amino acid residues. In aspects, the disordered N-terminal domain of Galectin-3 comprises from 1 to about 50 contiguous amino acid residues. In aspects, the disordered N-terminal domain of Galectin-3 comprises from 1 to about 30 contiguous amino acid residues. In aspects, the disordered N-terminal domain of Galectin-3 comprises from 1 to about 30 contiguous amino acid residues. In aspects, the disordered N-terminal domain of Galectin-3 comprises from 1 to about 20 contiguous amino acid residues. In aspects, the disordered N-terminal domain of Galectin-3 comprises from 1 to about 10 contiguous amino acid residues. In aspects, the disordered N-terminal domain of Galectin-3 is SEQ ID NO:11. In aspects, the disclosure provides a complex comprising Galectin-3 and a compound that binds to the disordered N-terminal domain of Galectin-3 and the allosteric cavity in the ordered C-terminal domain of Galectin-3 (including all aspects thereof, as described herein).

The disclosure provides methods for treating a disease characterized by overexpression or inappropriate expression of Galectin-3 in a subject in need thereof, the method comprising administering to the subject an effective amount of a compound; wherein the compound that is capable of inhibiting an interaction between an amino acid in the disordered N-terminal domain of Galectin-3 and the allosteric cavity in the ordered C-terminal domain of Galectin-3 (as described herein, including all embodiments thereof). In aspects, the disclosure provides methods for treating a disease characterized by overexpression or inappropriate expression of Galectin-3 in a subject in need thereof, the method comprising administering to the subject an effective amount of a compound; wherein the compound that is capable of inhibiting an interaction between Y36 and/or Y45 in the disordered N-terminal domain of Galectin-3 and the allosteric cavity in the ordered C-terminal domain of Galectin-3. In aspects, the compound that is capable of inhibiting an interaction between Y36 in the disordered N-terminal domain of Galectin-3 and the allosteric cavity in the ordered C-terminal domain of Galectin-3. In aspects, the compound that is capable of inhibiting an interaction between Y45 in the disordered N-terminal domain of Galectin-3 and the allosteric cavity in the carbohydrate binding domain of Galectin-3. In aspects, the compound that is capable of inhibiting an interaction between Y36 and Y45 in the disordered N-terminal domain of Galectin-3 and the allosteric cavity in the carbohydrate binding domain of Galectin-3. In aspects, the disclosure provides a complex comprising Galectin-3 and a compound that binds to Y36 and/or Y45 in the disordered N-terminal domain of Galectin-3 and the allosteric cavity in the C-terminal domain of Galectin-3 (including all aspects thereof, as described herein). In aspects, the allosteric cavity in the C-terminal domain of Galectin-3 is the F-face (or allosteric F-face) of the C-terminal domain of Galectin-3. In aspects, the compound is a peptide, a small molecule, or a macrocycle. In aspects, the compound has an inhibitor effect on Galectin-3 that is the same as or better than the peptide comprising the amino acid sequence of SEQ ID NO:9 and/or that fills the same space as the peptide comprising the amino acid sequence of SEQ ID NO:9. In aspects, the compound is a peptide, a small molecule, or a macrocycle.

In aspects, the disease characterized by overexpression or inappropriate expression of Galectin-3 is cancer, fibrosis, a cardiovascular disease, an infectious disease, an inflammatory disease, or a neurological disease. In aspects, the disease characterized by overexpression or inappropriate expression of Galectin-3 is cancer. In aspects, the disease characterized by overexpression or inappropriate expression of Galectin-3 is leukemia, ovarian cancer, breast cancer, bladder cancer, gastric cancer, prostate cancer, lung cancer, pancreatic cancer, thyroid cancer, colon cancer, melanoma, or lymphoma. In aspects, the disease characterized by overexpression or inappropriate expression of Galectin-3 is leukemia. In aspects, the disease characterized by overexpression or inappropriate expression of Galectin-3 is acute lymphoblastic leukemia. In aspects, the disease characterized by overexpression or inappropriate expression of Galectin-3 is fibrosis. In aspects, the disease characterized by overexpression or inappropriate expression of Galectin-3 is a cardiovascular disease. In aspects, the cardiovascular disease is heart failure. In aspects, the cardiovascular disease is atherosclerosis. In aspects, the disease characterized by overexpression or inappropriate expression of Galectin-3 is an infectious disease. In aspects, the infectious disease is meningitis. In aspects, the infectious disease is a coronavirus infection (e.g., SARS-CoV-1, SARS-CoV-2, MERS-CoV). In aspects, the infectious disease is COVID-19. In aspects, the disease characterized by overexpression or inappropriate expression of Galectin-3 is an inflammatory disease. In aspects, the inflammatory disease is type 1 diabetes. In aspects, the inflammatory disease is type 2 diabetes. In aspects, the inflammatory disease is sepsis. In aspects, the inflammatory disease is acute respiratory distress syndrome. In aspects, the inflammatory disease is caused by degradation of retinal ganglion cells, which can lead to optic nerve injury, retinal ischemia, or glaucoma. In aspects, the disease characterized by overexpression or inappropriate expression of Galectin-3 is a neurological disease. In aspects, the neurological disease is Alzheimer's disease. In aspects, the disease is caused by overexpression of Galectin-3. In aspects, the disease is caused by inappropriate expression of Galectin-3.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound, peptide, pharmaceutical composition, or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound, peptide, pharmaceutical composition, or method provided herein include cancer of the thyroid, endocrine system, brain, breast, cervix, colon, head and neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute lymphoblastic leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “cardiovascular disease” is used in accordance with its plain ordinary meaning. In aspects, cardiovascular diseases that may be treated with a peptide, compound, pharmaceutical composition, or method described herein include, but are not limited to, stroke, heart failure, hypertension, atherosclerosis, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis. In aspects, the cardiovascular disease is heart failure. In aspects, the cardiovascular disease is atherosclerosis.

The term “inflammatory disease” refers to a disease or condition characterized by aberrant inflammation (e.g. an increased level of inflammation compared to a control such as a healthy person not suffering from a disease). Examples of inflammatory diseases include acute respiratory distress syndrome, sepsis, autoimmune diseases, arthritis, rheumatoid arthritis, psoriatic arthritis, juvenile idiopathic arthritis, multiple sclerosis, systemic lupus erythematosus, myasthenia gravis, diabetes mellitus type 1 (i.e., type 1 diabetes), diabetes mellitus type 2 (i.e., type 2 diabetes), graft-versus-host disease, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. In aspects, the inflammatory disease is diabetes. In aspects, the inflammatory disease is type 1 diabetes. In aspects, the inflammatory disease is type 2 diabetes. In aspects, the inflammatory disease is sepsis. In aspects, the inflammatory disease is acute respiratory distress syndrome. In aspects, the inflammatory disease is caused by degradation of retinal ganglion cells, which can lead to optic nerve injury, retinal ischemia, or glaucoma.

The term “neurological disease” or “neurodegenerative disease” refers to a disease or condition in which the function of a subject's nervous system becomes impaired. Examples of neurodegenerative diseases that may be treated with a peptide, compound, pharmaceutical composition, or method described herein include Alexander's disease, Alper's disease, Alzheimer's disease, amyotrophic lateral sclerosis, ataxia telangiectasia, Batten disease (also known as Spielmeyer-Vogt-Sjogren-Batten disease), bovine spongiform encephaloopathy (BSE), Canavan disease, chronic fatigue syndrome, cockayne syndrome, corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, lewy body dementia, Machado-Joseph disease (Spinocerebellar ataxia type 3), multiple sclerosis, multiple system atrophy, myalgic encephalomyelitis, narcolepsy, neuroborreliosis, Parkinson's disease, Pelizaeus-Merzbacher Disease, Pick's disease, primary lateral sclerosis, prion diseases, Refsum's disease, Sandhoffs disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, progressive supranuclear palsy, or tabes dorsalis. In aspects, the neurological disease is Alzheimer's disease.

The term “infectious disease” refers to a disease or condition that can be caused by organisms such as a bacterium, virus, fungi or any other pathogenic microbial agents. In aspects, the infectious disease is caused by a pathogenic bacteria. Pathogenic bacteria are bacteria which cause diseases (e.g., in humans). In aspects, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In aspects, the disease is bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); bacterial meningitis (i.e. a bacterial inflammation of the meninges); bacterial pneumonia (i.e. a bacterial infection of the lungs); urinary tract infection; bacterial gastroenteritis; or bacterial skin infections (e.g. impetigo, or cellulitis). In aspects, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection. In aspects, the infectious disease is meningitis. In aspects, the infectious disease is a coronavirus infection (e.g., SARS-CoV-1, SARS-CoV-2, MERS-CoV). In aspects, the infectious disease is COVID-19.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In aspects, treating is preventing. In aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things. Treatment may also include supporting or enhancing the effects of standard-of-care or experimental clinical treatments, and mitigating deleterious effects of standard-of-care or experimental clinical treatment.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In aspects, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, and other non-mammalian animals. In aspects, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In aspects, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the peptides, compounds, and compositions can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Dose and Dosing Regimens

The dosage and frequency (single or multiple doses) of the Galectin-3 inhibitor (e.g., peptide, compound, or pharmaceutical composition described herein, including embodiments and aspects thereof) administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and Galectin-3 inhibitors described herein. Adjustment and manipulation of established dosages (e.g., frequency and duration) are well within the ability of those skilled in the art.

For any composition and Galectin-3 inhibitor described herein, the therapeutically effective amount can be initially determined from cell culture assays. Target concentrations will be those concentrations of Galectin-3 inhibitor that are capable of achieving the methods described herein, as measured using the methods described herein or known in the art. As is well known in the art, effective amounts of Galectin-3 inhibitor for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the Galectin-3 inhibitor may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the Galectin-3 inhibitor. Thereafter, the dosage is increased by small increments until the optimum effect under circumstances is reached. Dosage amounts and intervals can be adjusted individually to provide levels of the Galectin-3 inhibitor effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of Galectin-3 inhibitor by considering factors such as compound potency, relative bioavailability, patient body weight, presence and severity of adverse side effects.

Additional Therapeutic Agents

In the provided methods of treatment, additional therapeutic agents can be used that are suitable to the disease (e.g., cancer) being treated. Thus, in aspects, the provided methods of treatment further include administering a third therapeutic agent to the subject. Suitable additional therapeutic agents include, but are not limited to analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

Informal Sequence Listing

For SEQ ID NOS:1-9 and 13-15, the N-terminus can be an amide or the N-terminus can be a capped-amide. In aspects, the N-terminal capped-amide is an acetyl-capped amide (e.g., ACE). For SEQ ID NOS:1-9 and 13-15, the C-terminus can be a carboxyl group or the C-terminus can be a capped-carboxyl group. In aspects, the C-terminus capped-carboxyl group is an amide-capped carboxy group.

SEQ ID NO: 1 = Peptide 1
ARAMGYPGASY
SEQ ID NO: 2 = Peptide 2
ARAFGYPIYSY
SEQ ID NO: 3 = Peptide 3
YYPGAYPRRYR
SEQ ID NO: 4
AMAMGYPRASY
SEQ ID NO: 5
AMARGYPWYSY
SEQ ID NO: 6
SYMRAYPMQIP
SEQ ID NO: 7
SYMRAYPMQMP
SEQ ID NO: 8
YYPGAYPMRFR
SEQ ID NO: 9
AYPRRYR
SEQ ID NO: 10 = Galectin-3
MADNFSLHDA LSGSGNPNPQ GWPGAWGNQP AGAGGYPGAS
YPGAYPGQAP PGAYPGQAPP GAYPGAPGAY PGAPAPGVYP
GPPSGPGAYP SSGQPSATGA YPATGPYGAP AGPLIVPYNL
PLPGGVVPRM LITILGTVKP NANRIALDFQ RGNDVAFHFN
PRFNENNRRV IVCNTKLDNN WGREERQSVF PFESGKPFKI
QVLVEPDHFK VAVNDAHLLQ YNHRVKKLNE ISKLGISGDI
DLTSASYTMI
SEQ ID NO: 11 = N-terminal domain of Galectin-3
MADNFSLHDA LSGSGNPNPQ GWPGAWGNQP AGAGGYPGAS
YPGAYPGQAP PGAYPGQAPP GAYPGAPGAY PGAPAPGVYP
GPPSGPGAYP SSGQPSATGA
SEQ ID NO: 12 = C-terminal domain of Galectin-3
YPATGPYGAP AGPLIVPYNL PLPGGVVPRM LITILGTVKP
NANRIALDFQ RGNDVAFHFN PRFNENNRRV IVCNTKLDNN
WGREERQSVF PFESGKPFKI QVLVEPDHFK VAVNDAHLLQ
YNHRVKKLNE ISKLGISGDI DLTSASYTMI
SEQ ID NO: 13
ANTPCGPYTHDCPVKR
SEQ ID NO: 14
PTHVTCKYCPAGNRDP
SEQ ID NO: 15
PGAY

### EXAMPLES

The following examples are for purposes of illustration only and are not intended to limit the spirit or scope of the disclosure or claims.

### Example 1

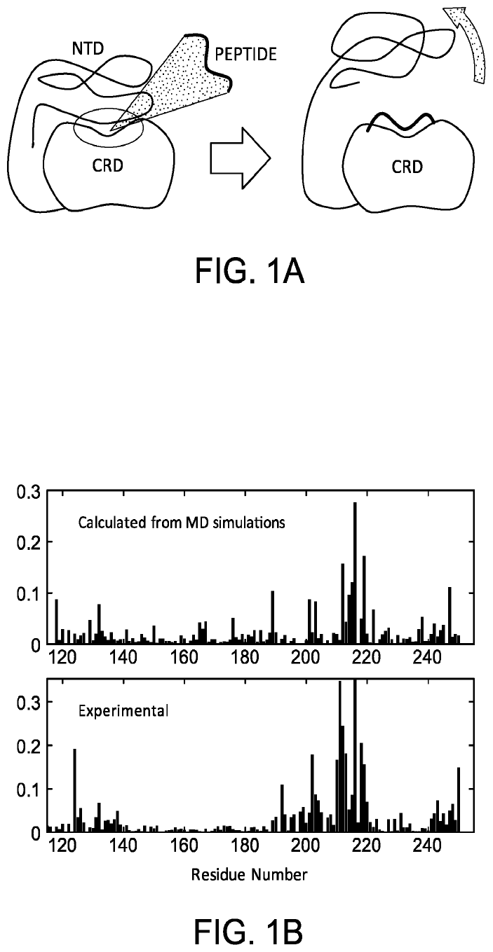

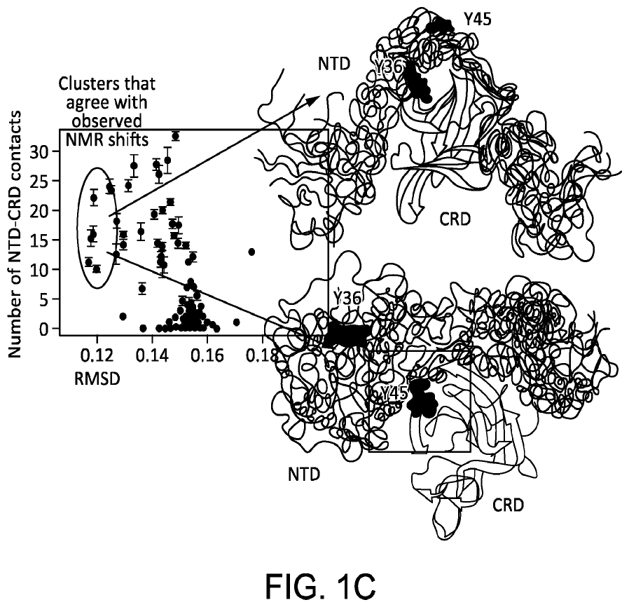

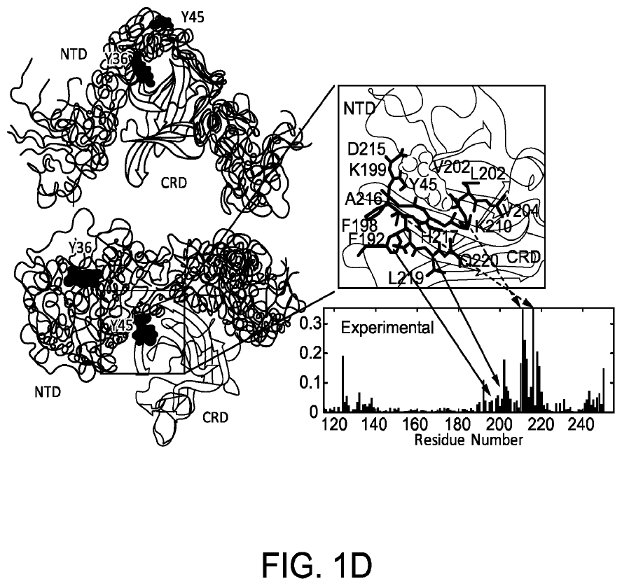

In order to overcome the problems in the art associated with Galectin-3 inhibitors, the inventors designed inhibitors that allosterically modulate the activity of Galectin-3 by binding a different region of the C-terminal domain which is far away from the lectin binding site. Importantly, the function of Galectin-3 is also modulated by the NTD through various mechanisms including phosphorylation and interaction with the CTD, as well as with other NTD in trans. Therefore, disrupting the interaction of the NTD with the CTD using designed inhibitors would also lead to the inhibition of Galectin-3 function (FIG. 1A). The challenge involved in designing such inhibitors stems from the lack of structure and the highly dynamic nature of the NTD. To overcome such challenges, the inventors developed a novel protocol combining nuclear magnetic resonance (NMR) data from recombinant Galectin-3 with enhanced molecular dynamic simulations, more particularly accelerated molecular dynamics (AMD) simulations, and in-silico peptide design methods (FIG. 2). Three designed peptides were tested in a Galectin-3 mediated agglutination assay. One was discovered to inhibit Galectin-3 agglutination, at a concentration comparable to the commercial Galectin-3 inhibitor TD-139, i.e., CAS Number 1450824-22-2 or 3-deoxy-3-[4-(3-fluorophenyl)-1H-1,2,3-triazol-1-yl]-β-D-galactopyranosyl 3-deoxy-3-[4-(3-fluorophenyl)-1H-1,2,3-triazol-1-yl]-1-thio-β-D-galactopyranoside.

Computational Design of Galectin-3 Inhibitors

The inventors designed peptides and small molecules that would inhibit Galectin-3 function by disrupting the interaction of the NTD with the CTD. The key step in such a design process was to obtain the ensemble of NTD conformations that interacted with the CTD under physiological conditions. Since the NTD is an intrinsically disordered region (IDR), it adopts multiple conformations under physiological conditions and is also highly dynamic. Therefore, methods such as X-ray crystallography that are typically used for determining protein structures are not applicable to IDRs. NMR spectroscopy can give structural information about IDRs in the form of peak intensities for individual residues in the amino acid sequence. However, NMR does not directly provide the 3D structural coordinates of the protein atoms, which are necessary for inhibitor design, unless the NMR data is interpreted using a predetermined protein structural ensemble generated in-silico. To generate the in-silico structural ensemble, MD simulations and Monte Carlo sampling of backbone dihedrals are typically used, but each of these methods suffers from their own deficiencies. Due to the vast protein conformational space, Monte Carlo-based methods may not be able to sample all the relevant conformations in reasonable time, whereas all atom MD simulations can only sample conformations that are accessible over a timescale of nanoseconds to low microseconds. IDR conformational transitions may span a timescale of hundreds of microseconds to milliseconds, which are beyond the reach of conventional MD simulations. Thus, it is challenging to generate an IDR structural ensemble using in-silico methods, which will cover the physiological IDR conformations. Thus, the challenges involved in the inhibitor design include: (1) generating a structural ensemble of the NTD-CTD complex using in-silico methods that include the physiological NTD conformations; (2) detecting the physiological NTD conformations from the very large in-silico ensemble using experimental information such as NMR, and (3) accounting for the dynamic nature of the NTD in the inhibitor design protocol; i.e. to be effective, the designed inhibitors should be able to disrupt the interactions of multiple structurally diverse NTD conformations binding to the CTD.

Derivation of the Galectin-3 NTD Ensemble and Initial Peptide Templates

To address the above challenges, the inventors developed a computational pipeline incorporating state-of-the-art MD simulation methods and in-silico peptide design algorithms. To address the problem of IDR conformational sampling, an enhanced MD method called accelerated MD (AMD) was used (Hamelberg et al., 2004). Using energy rescaling, AMD is capable of accessing timescales in the order of milliseconds, that are beyond the reach of conventional MD. Starting from an initial Galectin-3 structure, where the CTD was modeled based on an existing crystal structure and the NTD was modeled as a random polymer chain, AMD was used to generate the initial conformational ensemble having 50,000 NTD conformations. For each of these conformations, the corresponding chemical shifts were predicted using the software SHIFTX2, for both the full length protein as well as for the CTD alone (Han et al., 2011). The chemical shift differences (CSDs) were then calculated according to the formula: Δδ ppm=[(Δ¹H)²+(0.25Δ¹⁵N)²]^{1/2}, where Δ¹⁵N and Δ¹H are the chemical shift differences of the ¹⁵N labeled backbone nitrogen and hydrogen atoms between full length and CTD-only Galectin-3. The NTD conformations were clustered by their structural similarity and for each cluster, the root mean square deviation (RMSD) from the experimental NMR CSDs was calculated. The NMR data was published previously (Ippel et al., 2016). The clusters showing low CSD RMSD and a high number of NTD-CTD contacts (total 1300 conformations) were selected for further processing. The selected clusters are highlighted in FIG. 1C and the agreement with the experimental CSDs is shown in FIG. 1B.

By analyzing the NTD conformations that showed agreement with the experimental NMR data, two major classes of NTD-CTD contacts were identified, where Y36 and Y45 of NTD made contact within an allosteric cavity in the ordered C-terminal domain as shown in FIG. 1D. The inventors therefore envisioned that targeting this pocket with peptides and small molecules could inhibit the binding of the NTD. To design the inhibitory peptides, a few backbone templates were initially selected based on the ensemble of NTD conformations that showed agreement with NMR. The NTD conformations were clustered by similarity and the representative NTD conformations from the most populated clusters were selected for template design. For each selected NTD conformation, 5 residues on each side of Y36 or Y45 were retained as part of the template. In total, 4 different peptide templates were considered for the in-silico design. The main steps involved in obtaining the peptide templates from the Galectin-3 NTD ensemble are explained in FIG. 2A.

Computational Design of Inhibitory Peptide Sequences

Starting from a given peptide template, each residue was systematically mutated to all 20 amino acids and an affinity score was calculated using the software Maestro™ (Schrodinger LLC.), which represented the improvement in affinity of the mutant peptide over the starting NTD sequence. The top scoring mutations were analyzed to identify 2-3 positions in each template that were most amenable to mutagenesis. These positions were then mutated combinatorically to generate multiple double and triple mutants, and the top mutants by affinity score were analyzed for features such as strong interaction with the CTD hydrophobic cavity, low desolvation energy and sequence diversity. This step generated 8 peptide candidates, which were then subjected to 500 ns of all atom MD simulations in an explicit water environment, to test their stability of binding to the CTD. Also, the binding free energies were calculated using the MM-GBSA method (FIG. 2C and Table 1). During MD, four of the eight peptides left the CTD cavity within 300 ns and were deemed unstable (FIG. 2D). Among the rest which remained bound (these also showed strong interaction with the CTD as measured by the protein-peptide energy and number of hydrogen bonds), one peptide Y45_cls70_Y1_M8_R9_F10_R11 was very similar in sequence to another peptide in the list and hence eliminated. The other three peptides were subjected to experimental testing. The main steps in selecting the top peptide candidates starting with the NTD templates are described in FIG. 2B.

Table 1 shows the binding properties for eight candidate peptides calculated from all-atom MD simulations. Peptides 1, 2, and 3 (SEQ ID NOS: 1, 2, and 3, respectively) are the best binders according to their duration of binding to the CTD, number of protein-peptide hydrogen bonds and binding free energy. Peptide 3 (SEQ ID NO:3) was found to be a positive hit in the agglutination assay. In Table 1, Column A is stable binding duration (ns); Column B is binding free energy (kcal/mol); Column C is SEM; Column D is protein-peptide interaction energy (kcal/mol); Column E is desolvation energy (kcal/mol); Column F refers to the SEQ ID NO; and Column G is the number of stable peptide-protein H bonds.

TABLE 1

| Peptide | A | B | C | D | E | F | G | |
|---|---|---|---|---|---|---|---|---|
| Y36_cls3_M2_M4_R8 | 278 | −25.9 | 0.06 | −85.6 | 59.7 | 4 | 0 | |
| Y36_cls3_R2_M4 | 394 | −46.2 | 0.06 | −140.4 | 94.2 | 1 | 2 | Peptide_1 |
| Y36_cls5_M2_R4_W8_Y9 | 184 | −22.3 | 0.08 | −64.1 | 41.8 | 5 | 0 | |
| Y36_cls5_R2_F4_I8_Y9 | 500 | −42.3 | 0.06 | −124.5 | 82.2 | 2 | 1 | Peptide_2 |
| Y45_cls1_M3_R4_M8_I10 | 172 | −16.2 | 0.06 | −66.2 | 50 | 6 | 0 | |
| Y45_cls1_M3_R4_M8_M10 | 91 | −35.5 | 0.2 | −87.8 | 52.3 | 7 | 0 | |
| Y45_cls70_Y1_M8_R9_F10_R11 | 500 | −34.4 | 0.5 | −136.6 | 102.2 | 8 | 0 | |
| Y45_cls70_Y1_R8_R9_Y10_R11 | 500 | −29.5 | 0.04 | −169.3 | 139.8 | 3 | 2 | Peptide_3 |

### Example 2

The NMR-based chemical shift differences measured by Ippel et al (25) between full-length and CTD-only Galectin-3 provided information about the dynamics, but these are averaged values and do not inform on individual structures. However, it is likely that the Galectin-3 IDR will adopt an ensemble of structurally diverse conformations, that transition in the picosecond to millisecond timescale under physiological conditions and which poses serious challenges to the application of computational methods. Here, we have approached the general problem of IDR characterization using accelerated molecular dynamics (AMD) combined with existing structural data of Galectin-3 to predict the binding interface of the CTD with the IDR. The CTD binding/N-terminal interface, as observed in the AMD simulations, includes a diverse ensemble of structures in which multiple amino acid motifs between residues 20-100 of Galectin-3 engage with the CTD. We show that these structures collectively explain the NMR data from Ippel et al (25) and agree with the fuzzy complex model of IDR interaction. In-silico designed peptides based on the interacting N-terminal motifs were then used to validate the model predicted by AMD. The process described here could be used to economically target other IDR interactions with proteins or protein domains with defined structures.

Materials and Methods

Molecular modeling. We retrieved the human Galectin-3 CTD crystal structure from the PDB Databank (PDB ID: 6FOF) (28). The NTD was added as a random chain using Modeller (29). The full-length structure was subjected to 100 ns of MD simulation in an implicit solvent environment (30). The protein conformations were then clustered by backbone RMSD and the mean radius of gyration was calculated for each cluster. We selected a representative structure from the cluster of which the mean radius of gyration was closest to the experimentally measured one for Galectin-3 (26). This structure was used as the starting conformation for the AMD simulations. The starting structure for the AMD was solvated in explicit water and ions were added to neutralize the net charge. The system was parameterized using the a99sb-disp force field, which has been shown to perform well with both folded and disordered proteins (31). Since Galectin-3 consists of both a folded and a disordered domain, this force field is a suitable choice. Further, hydrogen mass repartitioning was implemented in order to use a 4 fs timestep (32). The system was first heated at constant volume from OK to 310K over 30 ns with harmonic restraints applied to the protein heavy atoms. Then the system was equilibrated for 50 ns in the NPT ensemble, while the heavy atom restraints were gradually reduced to zero. Finally, the system was equilibrated for a further 50 ns unrestrained.

Five independent AMD simulations at 310K (NPT ensemble), each lasting for 250 ns were performed using the GPU accelerated AMBER software package (33). The Galectin-3 NTD conformations resulting from the five simulations were clustered by backbone dihedral RMSD and for each cluster, the average number of NTD-CTD contacts was determined. Two residues of which the Cα atoms were within 8.5 Å were defined as a contact. We also calculated the average per residue chemical shift difference (Δδ) for each cluster using the software SHIFTX2 (34). For calculating Δδ, we calculated the chemical shifts for the full-length Galectin-3 and those of the CTD domain only by truncating the NTD region. The Δδ was then obtained as the difference between the two shifts as in Ippel et al (25). Finally, the RMSD between the calculated and experimental Δδ was determined for each cluster and plotted against the number of NTD-CTD contacts (FIGS. 1C-1D). The clusters with the lowest Δδ RMSD as well as with average number of NTD-CTD contacts greater than five (circled clusters in FIGS. 1C-1D) were combined to obtain a conformational ensemble with significant NTD-CTD interactions, and that is in agreement with experimental NMR data.

Bayesian maximum entropy method. The details of the BME approach is described in (35). Briefly, the weights for the AMD derived protein ensemble ([w₁ . . . wₙ], n: total number of conformations) were obtained by minimizing the cost function

$$L ( {w}_{1} … {w}_{n} ) = \frac{m}{2} {χ}^{2} ( {w}_{1} … {w}_{n} ) - θ {S}_{R E L} ( {w}_{1} … {w}_{n} ) , where {χ}^{2} ( {w}_{1} … {w}_{n} ) = \frac{1}{m} {\sum }_{i}^{m} \frac{{( {\sum }_{j}^{n} {w}_{j} F ( {x}_{j} ) - {F}_{i}^{E X P} )}^{2}}{{σ}_{i}^{2}}$$

is the agreement between observed and experimental CSDs and

$${S}_{R E L} = - {\sum }_{j}^{n} {w}_{j} log ( \frac{{w}_{j}}{{w}_{j}^{0}} )$$

is the entropy relative to starting weights. Here, xⱼ denotes the set of protein coordinates for the j^{th} conformation, F(xⱼ) represents the calculated CSDs using SHIFTX2 and Fᵢ^{EXP} is the CSD for the i^{th} residue. m is the number of residues for which experimental CSDs are available. Initially, all conformations were assigned the same weight w₀, where w₀=I/n. σᵢ denotes the uncertainty of SHIFTX2 in calculating the ¹H and ¹⁵N CSDs from structure and are obtained from (34). θ is an adjustable parameter that determines the tradeoff between the entropy and the agreement with experiments. The optimal value of θ was determined by performing the optimization for different values of θ and locating the elbow of the χ² vs. log log₁₀(θ) curve, as suggested by (35). The optimization was carried out using the ‘stats’ package in R.

Cells, culture and agglutination assay. LAX56 human pre-B ALL cells were routinely co-cultured with mitomycin-C inactivated OP9 stromal cells. These previously described primary leukemia cell grew directly out from a relapse bone marrow sample (36, 37). For agglutination assays, cells were harvested, washed once in α-MEM medium, resuspended in 10 ml X-VIVO 15 medium (Lonza) and incubated at 37° C. for 24 h remove the Galectin-3 produced by OP9 stromal cells. For the assay, ALL cells were resuspended in X-VIVO 15 medium at a concentration of 1×10⁶/ml and seeded at 2×10⁵/200 μl cells into wells. GST (12.5 μg/ml) or GST-Galectin-3 (25 μg/ml or 150 μg/ml, two different isolates) recombinant proteins were added in 300 μl X-VIVO 15 medium to duplicate wells. Peptides, if included, were preincubated for 5 minutes with the recombinant proteins and added at different concentrations as indicated in the figures. TD139 was purchased from MedChemExpress and used at 100 μM. Phase contrast images were taken after 1-2 hours. Agglutination was defined as aggregates containing >10 cells per cluster. 2-13 images from different areas were taken and evaluated for cell clusters per condition. Biological data were graphed with GraphPad Prism software (version8.3.1). Values represent mean±SEM of the number of aggregates scored per independent image.

GST-Galectin-3 and mutants. Full-length human GST-Galectin-3 (hereafter named GST-Gal3) in pGEX2T was previously described (38). To generate mutants, we used Takara on-line primer design tools and a Takara In-Fusion HD Cloning Plus kit including CloneAmp HiFi PCR and Takara PCR enhancer to generate mutations according to the manufacturer's instructions. DNAs run on agarose gels were purified using a Thermo Scientific GeneJET Gel Extraction Kit (Cat #K0691). In-Fusion reactions (Takara) were assembled and Thermo Scientific™ BL21(DE3) competent cells used for transformation. All constructs were verified by DNA sequencing (Eton Bioscience, San Diego, Calif.).

Galectin-3 CTD construct for NMR. The Galectin-3 C-terminal domain construct was generated using the same methods described above for the mutants. The protein includes Galectin-3 amino acids P117-1250 as well as residual attached glycine and serine residues after thrombin cleavage. Single colonies were grown overnight in LB+ amp, collected by centrifugation then inoculated in M9 media with ammonium-¹⁵N chloride (Sigma) and grown for 3-4 hours. After induction of protein production with IPTG for an 3-4 additional hrs, cells were harvested and suspended in 1% NP40, PI, PMSF, 1 mM DTT, 50 mM Tris-HCl, pH 7.5. Cells were disrupted by sonication. GST-Galectin-3 was bound to glutathione-agarose (Genscript Cat #L00207) overnight at 4° C. Beads were washed 4× in lysis buffer, then suspended in 50 mM Tris-HCl pH 7.5, 0.1 mM DTT and treated with 60 u thrombin/ml (Fisher Cytiva Thrombin Protease) for 16 hrs at RT. The supernatant containing Galectin-3 protein was treated with benzamidine sepharose (Sigma, HiTrap Benzamidine FF) to bind and remove thrombin. Protein was concentrated using an Amicon 3K filter and used in 20 mM potassium phosphate buffer pH 6.8, 0.1 mM DTT for NMR. Protein concentrations were determined by BCA.

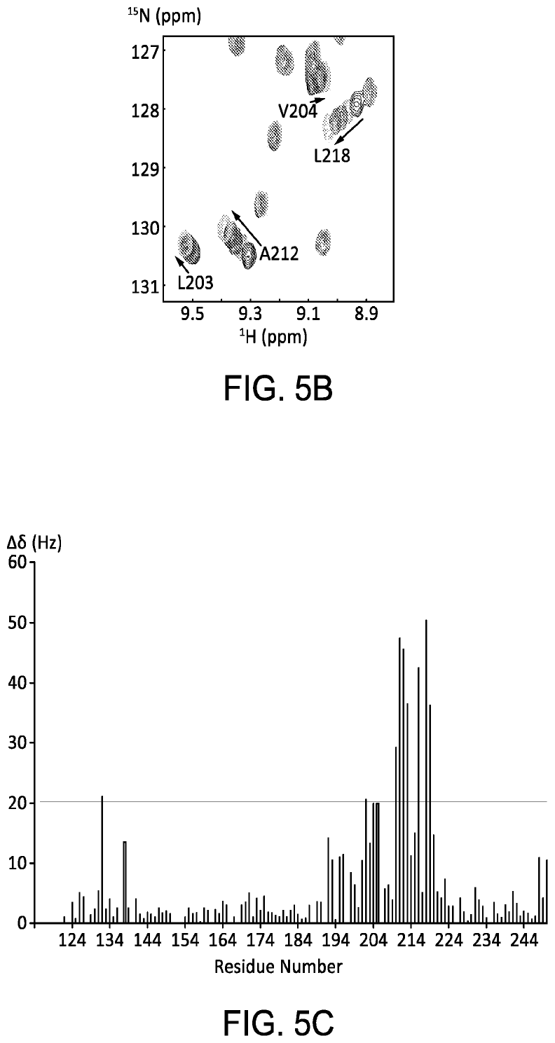

NMR. ¹⁵N-¹H HSQC 700 MHz spectra were acquired with 20 μM Galectin-3 CTD [in 20 mM potassium phosphate buffer, pH 6.8, 0.1 mM DTT] and different molar ratios of added peptide-3 as indicated in FIG. 5. The chemical shift perturbation (CSP) 06 was calculated using the following equation: Δ\ delta=\sqrt{(Δω_{N}²+Δω_{H}²)/2}. The Δω_{N} and Δω_{H} are the nitrogen and proton chemical shift difference between free ¹⁵N-CTD and that in the mixture with P3 peptide. Assignments are based on the Galectin-3 CTD NMR data of Ippel (25) and Umemoto (39). N-terminal domain sequences in our construct are slightly different from theirs, causing limited miss-assignment in the N-terminal domain, and ambiguity between residues 240-248 due to their close contact with the short β-strand in the slightly different N-terminal domains. Because L135 and W181 patterns differ between Ippel and Umemoto, their assignments could not be unambiguously determined. Also the position of T137 differs between Ippel and Umemoto, and position changes of T248 make its assignments unclear. However, none of these residues appear to be involved in the interaction with P3 peptide since their chemical shift perturbation was quite small, except for residue T248, with a CSP about 10.9 Hz and one unit of RMSD.

Peptides. Peptides were purified by HPLC. These included peptide-1 ACE-ARAMGYPGASY-NH₂ (SEQ ID NO:1), peptide-2 N-terminal acetyl-ARAFGYPIYSY-C-terminal amide and peptide-3 ACE-YYPGAYPRRYR-NH2 (SEQ ID NO:3). Peptide-4 was the Galectin-3 inhibitory peptide ANTPCGPYTHDCPVKR G3-C12 (SEQ ID NO:13) described in Zou et al (40) to target the CTD and peptide-5 the scrambled negative control peptide PTHVTCKYCPAGNRDP G3-H12s (SEQ ID NO:14) described in the same study. Peptide-4 and Peptide-5 did not have an effect on Galectin-3-mediated agglutination (data not shown).

Results

Derivation of the Galectin-3 NTD Ensemble

An enhanced MD method called accelerated MD (AMD) uses an innovative energy rescaling method to access timescales in the order of milliseconds, that are beyond the reach of conventional MD (41). Therefore, we applied AMD to the problem of IDR conformational sampling of the Galectin-3 NTD. Starting from the initial Galectin-3 structure, where the CTD was modeled based on an existing crystal structure and the NTD was modeled as a random polymer chain, AMD was used to generate the initial conformational ensemble consisting of 50,000 NTD conformations. For each of these conformations, the corresponding chemical shifts were predicted using SHIFTX2 software, for both the full length protein as well as for the CTD alone (34). The chemical shift differences (CSDs) were then calculated according to the formula Δδ ppm=[(Δ¹H)²+(0.25Δ¹⁵N)²]^{1/2}, where Δ¹⁵N and Δ¹H are the chemical shift differences of the ¹⁵N labeled backbone nitrogen and hydrogen atoms between full length and CTD-only Galectin-3 (FIG. 1B, top panel). The NTD conformations were clustered by their structural similarity (details in the methods section) and for each cluster, the root mean square deviation (RMSD) from the experimental NMR CSDs (25) were calculated. The clusters showing low CSD RMSD and a high number of NTD-CTD contacts, including a total of 1300 conformations, were selected for further processing (FIG. 1C). As shown in FIG. 1B there was an excellent agreement between the AMD-calculated and experimental CSDs within the filtered ensemble. The selected clusters are highlighted in FIG. 1C. By analyzing the NTD conformations that showed agreement with the experimental NMR data, two major classes of NTD-CTD contacts were identified, where of all residues, Y36 and Y45 of the NTD made the most long-term contact with the CTD, with a shallow cavity in the CTD as shown in FIGS. 1C-1D. The model predicted that the cavity would encompass candidate contacts including residues F192, F198, K199, Q201, V202, L203, V204, K210, D215, A216, H217, L219 and Q220. These residues that show close contact with the NTD in the MD ensemble also correspond to the strongest peaks in the experimental CSD profile, as shown in FIG. 1D.

Experimental Verification Approach

The AMD-generated model thus predicted critical regions of IDR-CTD contact that could involve a targetable pocket. We used two approaches to test this experimentally. Mutation of critical residues in that pocket could abolish binding to the IDR and the agglutination activity of Galectin-3. Also, a peptide could potentially fit in the shallow pocket and inhibit the IDR interaction. A classical test for carbohydrate-binding activity of a lectin including Galectin-3 is an agglutination assay (36, 38, 42). In this assay, recombinant Galectin-3 is tested for its ability to promote lattice formation by binding in a multivalent manner to glycoproteins located on the cell surface: when cell surface glycoprotein targets are located on different cells, carbohydrate binding combined with multimer formation causes cellular agglutination. Such an assay has widespread use for testing Galectin-3 inhibitors (e.g., 21, 40). Thus we used an agglutination assay in which recombinant Galectin-3 is added to patient-derived precursor B-acute lymphoblastic leukemia cells (pre-B ALL) as a readout for Galectin-3 lattice-forming activity.

Computational Design of Inhibitory Peptide Sequences

To design inhibitory peptides, a limited number of backbone templates were initially selected based on the ensemble of NTD conformations that showed agreement with NMR. The NTD conformations were clustered by similarity and the representative NTD conformations from the most populated clusters were selected for template design. The initial templates were obtained by retaining 5 amino acids on both sides of Y36 or Y45 in the CTD bound NTD conformations. The main steps involved in obtaining the peptide templates from the Galectin-3 NTD ensemble are shown in FIGS. 2A-2B. Starting from a given peptide template, each residue was systematically mutated to all 20 amino acids and an affinity score was calculated using Maestro™ software (Schrodinger LLC.), which represents the improvement in affinity of the mutant peptide over the starting NTD sequence (43). The top scoring mutations were analyzed to identify 2-3 positions in each template that were most amenable to mutagenesis. These positions were then mutated combinatorically to generate multiple double and triple mutants, and the top mutants by affinity score were analyzed for features such as strong interaction with the CTD hydrophobic cavity, low desolvation energy and sequence diversity. This step generated 8 peptide candidates, which were then subjected to 500 ns of all-atom MD simulations in an explicit water environment, to test their stability of binding to the CTD. Also, the binding free energies were calculated using the MM-PBSA method in the AMBER software package (44) (Table 1 and FIG. 2C). During MD, 4/8 peptides left the CTD cavity within 300 ns and were deemed unstable (Table 1 and FIG. 2D). Among the rest which remained bound and also showed strong interaction with the CTD as measured by the protein-peptide energy and number of hydrogen bonds, one peptide, Y45_cls70_Y1_M8_R9_F10_R11, was very similar in sequence to another peptide in the list and hence eliminated. The other three peptides were subjected to experimental testing (Table 1).

Peptide Testing on Pre-B ALL Cells

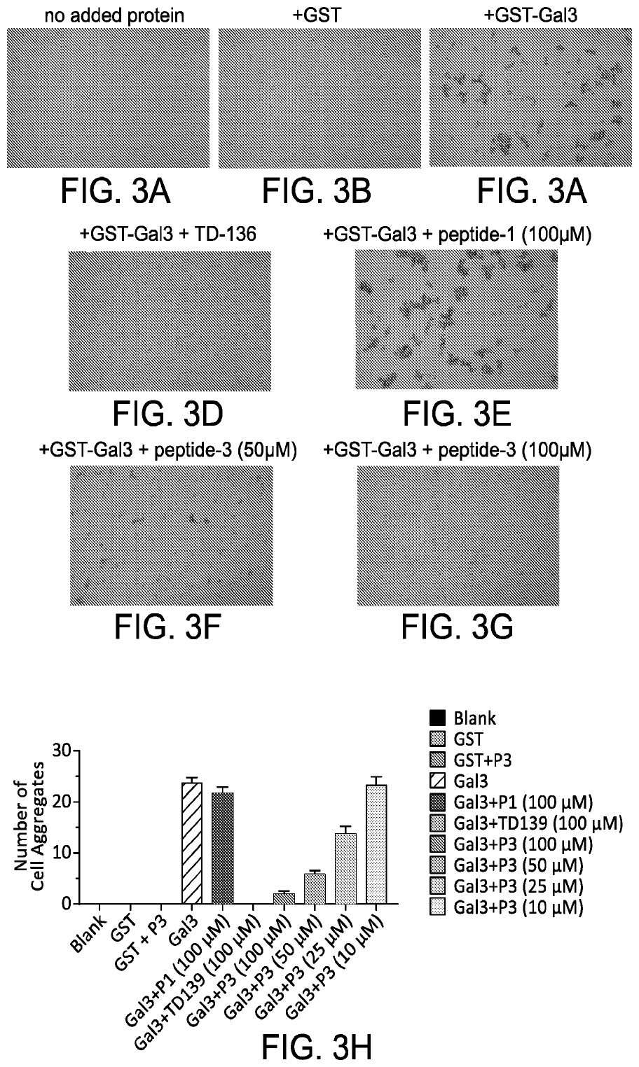

We tested these peptides in the agglutination assay. As shown in FIG. 3A, without treatment, LAX56 cells appear as a single-cell suspension. When GST alone was added as a negative control, (FIG. 3B) no agglutination was measured, whereas GST-Gal3 (FIG. 3C) caused cellular agglutination as expected. We used the glycomimetic (TD139 (45, 46) and citations therein) as positive control (47) and the compound clearly inhibited Galectin-3 mediated agglutination (FIG. 3D). Two of the three peptides tested, P1 peptide-1 (FIG. 3E) and P2 peptide-2 (not shown) had no effect on agglutination. However, peptide-3 (P3) clearly was inhibitory: there was a dose-response, with a correlation between different concentrations of P3 and degree of disruption of Galectin-3-mediated lattice formation (FIGS. 3F-3H).

Site-Directed Mutagenesis Identifies Residues Important for Galectin-3 Agglutination Function

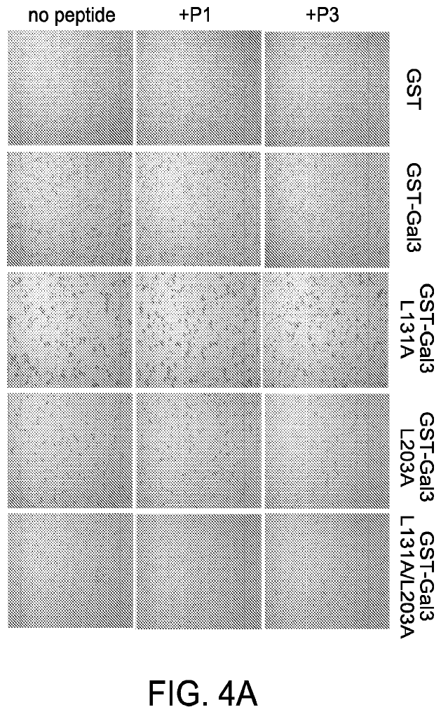

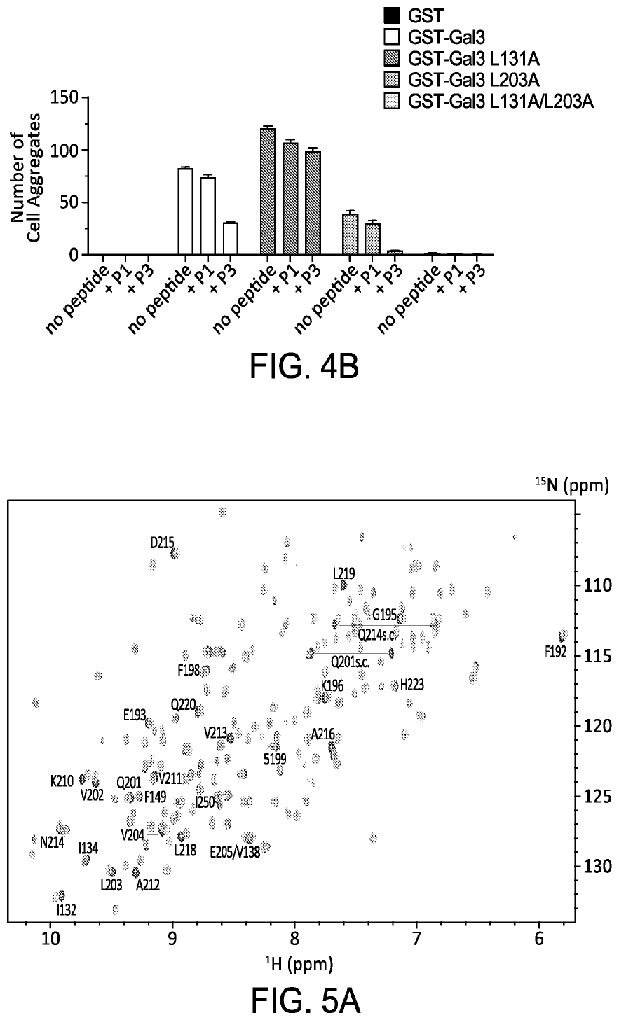

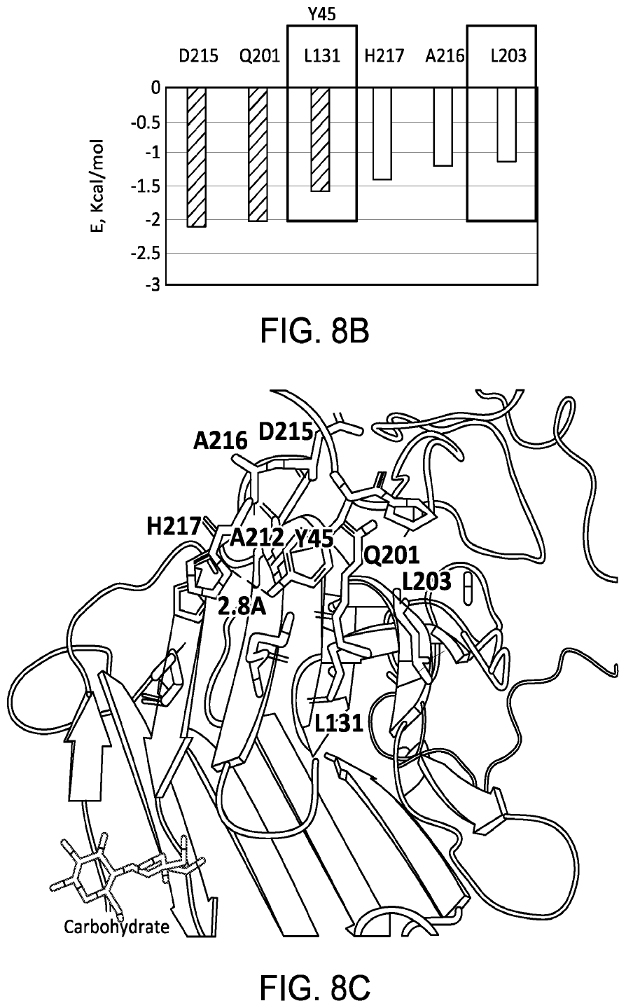

The model predicted strong contacts of, among others, Y36 and Y45 in the IDR with amino acids L131, L203 and H217 in the CTD (FIG. 8). Therefore, the latter three amino acids were mutated to alanine to test their contribution to the Galectin-3 agglutination activity. However GST-Gal3 L131A and GST-Gal3 L203A (FIG. 4A, left panel, FIG. 4B right panel quantitation) as well GST-Gal3 H217 (not shown) still were able to agglutinate LAX56 cells, although the ability of the L131A Gal3 mutant was enhanced, and that of L203A reduced, compared to wild type Gal3 (FIG. 4B). Moreover, the agglutination mediated by the L203A mutant could still be inhibited by P3 peptide-3, but interestingly the L131A mutant was largely insensitive to inhibition. We then generated a L131A/L203A double mutant. As shown in FIG. 4, this mutant was functionally inactive and failed to agglutinate LAX56 leukemia cells. This identified L131 and L203 as contact points of the IDR with the shallow pocket in the CTD that are essential for agglutination.

NMR Identifies Contacts of Peptide-3 with the F-Face of the CTD

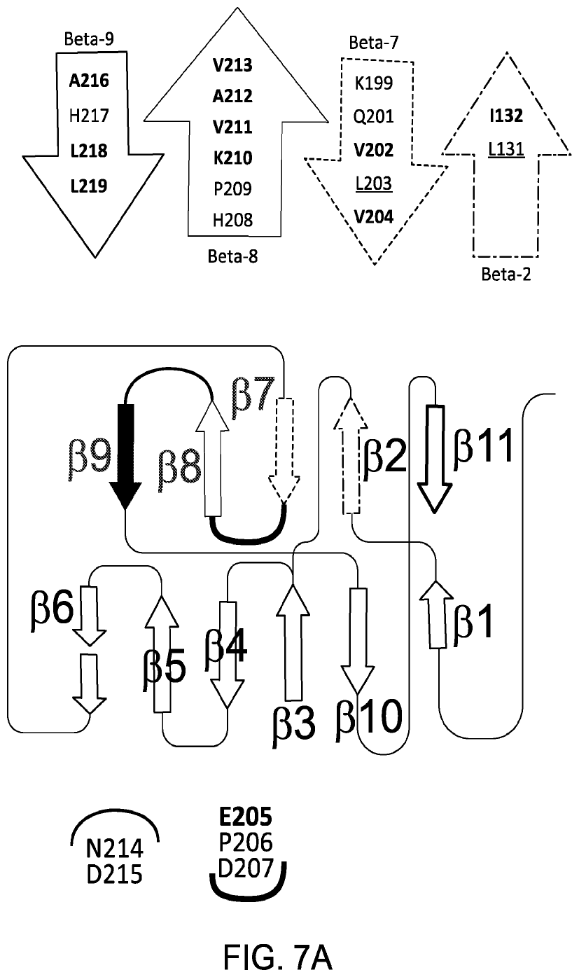

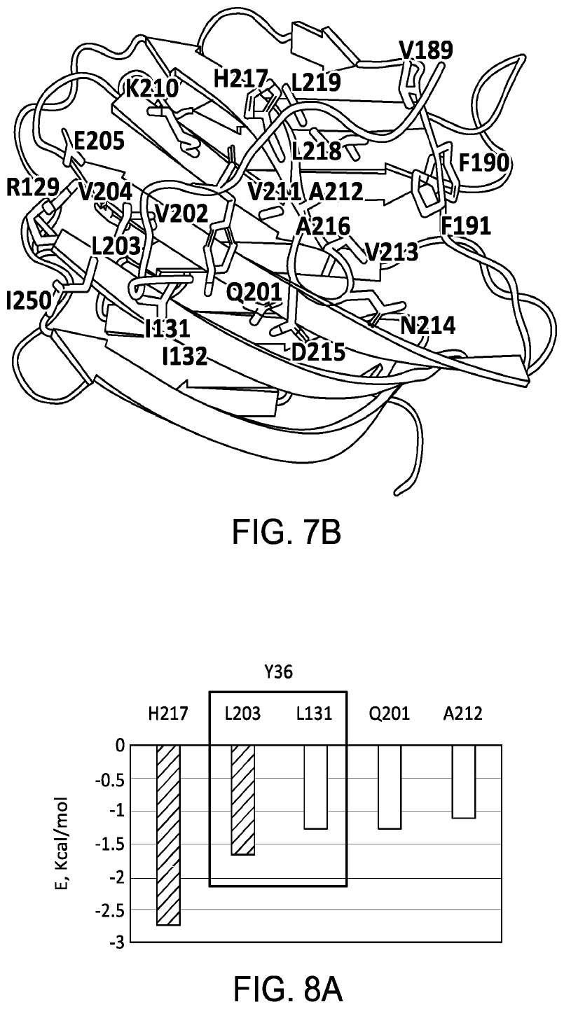

If P3 peptide-3 inhibits GST-Gal3 mediated agglutination by interfering with the interaction of the IDR with the CTD, P3 would likely make contact with the CTD. We next used NMR to investigate this. We generated a Galectin-3 CTD construct including amino acids 117-250 and used published NMR structure data (25, 39, 48, 49) for assignments of CTD amino acid residues. As shown in FIG. 5, NMR showed that P3 makes extensive contacts with amino acids in the CTD. In a dose-response titration with increasing concentrations of P3, as exemplified in FIG. 5B, large Δδ shifts were measured with a number of amino acids such as L203, V204, A212 and L218. FIG. 5C provides a summary of the chemical shift perturbation (CSP) measured at the highest molar ratio of Galectin-3 CTD to P3 for the amino acid residues identified. There were seven amino acids that had a more than two-fold increased RMSD of their CSP when exposed to P3. This included residues K210, V211, A212 and V213 in the (38 sheet as well as A216, L218 and L219 in the (39 sheet. Other residues with an increased RMSD of around 2 in their CSP included V202, V204 and E205 located in the (37 sheet, and I132 in the (32 sheet. These residues are all located on the F-face of the CDR (FIG. 7A). Residues such as R186, K227 or Y221 which are located in the S-face of the CTD exhibited no shift upon exposure to P3 (data not shown).

Bayesian Maximum Entropy (BME) Approach Uncovers Diverse CTD-Bound NTD Conformations

We also further investigated the NTD-CTD interaction obtained from AMD using the Bayesian maximum entropy (BME) method. The details of the BME approach are given in the methods section. In brief, the BME approach tries to achieve agreement between an MD-derived ensemble and available experimental data, while maximizing the information entropy within the obtained ensemble. This leads to a conformational ensemble that maintains its diversity, while still agreeing with the experimental data. The BME approach assigns a weight to each conformation, which is proportional to its contribution to the measured experimental property.

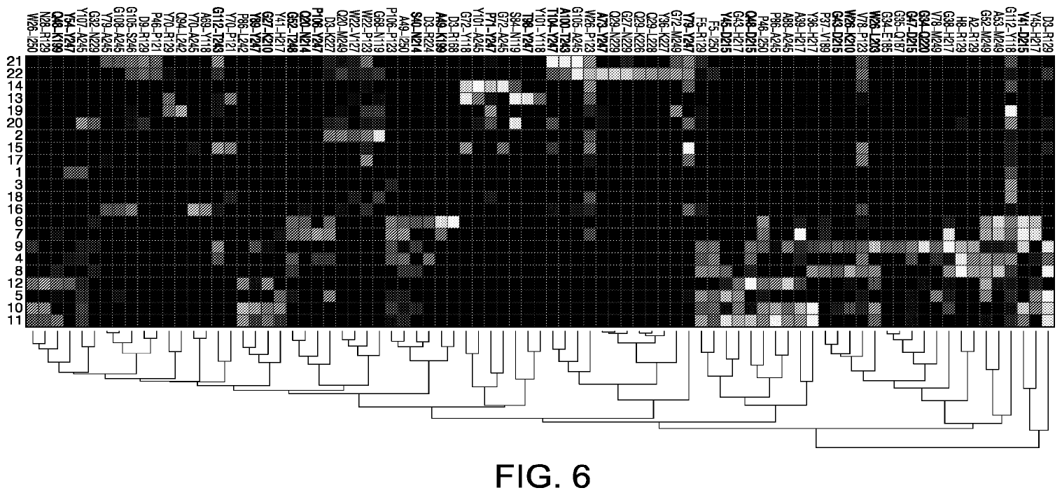

By applying the BME approach, and using the per residue CSDs as experimental data, we calculated the weight of each NTD conformation from AMD. The highest weighted conformations were then clustered by dihedral RMSD and within each cluster, the frequencies of pairwise residue contacts between the NTD and the CTD were obtained. FIG. 6 shows the normalized frequency of each inter-residue contact within the different clusters. Applying the BME approach, we therefore obtained a diverse ensemble, in which apart from Y36 and Y45, multiple NTD residues have significant interactions with the CTD. The contacts where the CTD residue shows a significant peak in the experimental CSD profile are highlighted in red in the heatmap. Interestingly, we find that multiple NTD residues, notably several aromatic residues such as W22, Y101, Y41, Y45, Y54, Y70, Y79 make contact with the CTD in a way that satisfies the NMR data. Also, looking at the pairwise interactions, it appears that in many cases, multiple NTD residues interact with a single CTD residue in different conformations. Examples of such contacts include those shown in Table 2 (i.e., one or more amino acids in the disordered N-terminal domain (NTD) make contact with the amino acid in the carbohydrate-recognition/binding domain (CTD)). Such interactions are indicative of the fuzzy interactions by IDPs that are widely addressed in the literature (50). The role of multiple aromatic residues in Galectin-3 mediated agglutination has recently been discussed (27). By exchanging among multiple NTD residues that interact with a localized CTD domain, Galectin-3 is able to minimize the loss of entropy upon binding. This is likely to lead to a more robust binding between the NTD and the CTD.

TABLE 2

| Amino Acid in Disordered | Amino Acid in Carbohydrate- |
|---|---|
| N-Terminal Domain | Recognition/Binding Domain |
| Y41/Y45/G47/Q48 | D215 |
| Y79/A73/T104/T98/P71/P106/Y89/Y54 | Y247 |
| A100/G112 | T243 |

### DISCUSSION

Over the past few decades, IDPs have emerged as critical proteins, playing major roles in various biochemical pathways. This creates unprecedented opportunities for using these proteins as drug targets. However, the three main challenges in designing drugs targeting IDPs using structure based approaches are (1) the lack of well-defined structure, (2) the difficulty to translate experimental structural information into three dimensional atomic coordinates, (3) lack of understanding of how conventional drug design approaches that target specific protein structures can be applied to the ensemble of diverse conformations of an IDP (51-54). Moreover, designing therapeutics necessitates a detailed mechanistic understanding of IDP dynamics and the interaction with self or other partners. Here, we have used Galectin-3 as a prototypical IDP to explore the potential of designing function-targeting therapeutics. We first analyzed the dynamics of the disordered NTD and it's interaction with the allosteric F-face of the CTD to gain mechanistic and structural insights into the dynamics of the disordered domain. Using the AMD method that efficiently samples the IDP conformational space, and existing NMR data that enables filtering the MD derived ensemble into an experimentally relevant subset, we identified diverse NTD conformations bound to the CTD. This is unprecedented, since the NMR data alone only allowed the identification of the CTD residues that interact with the NTD, but not the specific NTD structures that contribute to this interaction.

However, the latter information is key to designing any kind of therapeutics using structural approaches. We then used the interacting NTD conformations as templates and, using in silico mutation scanning, successfully designed a peptide that inhibited Galectin-3-mediated cellular agglutination. We further verified the predicted binding pose of the peptide in Galectin-3 using NMR experiments. Importantly, the specific chemical nature of this peptide and the interacting neighborhood within the IDP can be used to construct pharmacophores, which can then be searched against existing compound databases to increase the likelihood of finding small molecule inhibitors. Our work thus serves as a proof of concept for therapeutically targeting other IDPs as well.

Because Galectins have multiple contributions to, among others, cancer drug resistance and tumor progression (55, 56), drugs that could modulate their activities would be important novel additions to standard-of-care cancer therapy. Of the human lectins, Galectin-3 is arguably one of the most intensively studied proteins. Its small size of 26 kDa would appear to facilitate structure-activity relationship analysis. However, the NTD, which consists mainly of the IDR, has not been examined in as much detail as the structured CTD because of problems inherent to investigation of non-structured conformations. Here, we used an approach to examine the IDR of Galectin-3 that is essentially without bias: AMD first generated all possible conformations of this domain and then we used experimentally obtained chemical shift differences between full length Galectin-3 CTD and the CTD-only to winnow out structures that were compatible with those data.

This analysis showed that Y36 and Y45, of all the IDR residues, make the most stable contacts with the CTD. However, site-directed mutagenesis of Y36Δ and Y45Δ alone or in combination still yielded a Galectin-3 protein capable of agglutinating pre-B ALL cells (data not shown). This result is consistent with a previous study by Lin et al (26) using NTD-truncated constructs, who concluded that no single site of the NTD is critical for its interaction with the CTD, and those of Zhao et al (21) who mutated 14 prolines in the NTD. Besides our original RMSD based approach, we also used the Bayesian maximum entropy method to filter the AMD ensemble. The BME approach maximizes the diversity of the filtered ensemble, while still maintaining agreement with the NMR data. The NTD ensemble obtained through the BME method showed multiple aromatic residues in the NTD that interact with the CTD, apart from Y36 and Y45. Nonetheless, Y36 and Y45 were essential for the process of computational modeling that designated a shallow pocket as a possible area of contact and led to the novel identification of two amino acids in that pocket, L131/L203, that are essential for Galectin-3-mediated leukemia cell agglutination. L203 was previously shown to be important for the interaction between the NTD and CTD (25) and an L203A Galectin-3 mutant has reduced capacity to form liquid-liquid phase separation droplets (21). In concordance with this, we also found that the L203A single mutant had reduced ability to agglutinate the leukemia cells although it still retained some activity. To assess the impact of these two residues L131 and L203 on the NTD interaction, we calculated the average interaction energy of each CTD residue in the F face in the NMR filtered AMD ensemble. The top five CTD residues showing the lowest interaction energy are shown in FIG. 8. We calculated the interaction energy separately in the two conformational clusters, where either Y36 or Y45 makes contact with the CTD. In both cases, L131 and L203 show up at the top, along with several other polar residues such as H217, Q201 and D215. The impact of polar residues on protein-protein interaction are likely to be small due to competition with the solvent. This leaves L131 and L203 as the key hydrophobic residues that contribute to NTD binding, with an interaction energy of −1 to −1.5 kcal/mol. According to previous computational studies on PPI (protein-protein interface) hotspots, an energy contribution >−2 kcal/mol is likely to impact binding of partner proteins significantly (57). Here, individually, the energy contributions of the two hydrophobic residues are less than −2 kcal/mol, but together, their contribution is a substantial −2 to −2.5 kcal/mol. The importance of AMD analysis was thus illustrated by the additional identification of the need for cooperativity of L131 with L203 in Galectin-3 to allow this lectin to agglutinate leukemia cells.

Studies to inhibit Galectin-3 classically focused on blocking the binding of carbohydrate substrates to the recognition domain. Reagents that are based on such a mechanism of action include carbohydrate mimetics (14), peptides that bind to the CTD (40), and, recently, function blocking antibodies (58). However, because the NTD-CTD interaction appears to be essential for the extracellular activity of Galectin-3, the inhibition of this interaction may afford an alternative approach. This likely contributes to the mechanism of inhibitory action of galactomannins (59) and PTX008, a calixarene (48). The latter compound makes contact with residues in the F-face of the CTD including V202, K210, V211 and A216, which are also contacted by peptide-3 in our study. However, galactomannins and PTX008 may also make some contacts with the S-face of the CTD, and inhibit Galectin-1, a lectin that has overlap in binding targets with Galectin-3 (36, 60).

Here using an unbiased, entirely in silico approach, we identified a peptide with the sequence YYPGAYPRRYR (SEQ ID NO:3) that inhibits Galectin-3 mediated agglutination. This is a remarkable result because the computational approach reduced a large number of candidate peptides for experimental screening to a very small number and moreover, included the PGAY (SEQ ID NO:15) motif previously shown to be critical in the Galectin-3 NTD-CTD interaction (25). In that study, the PGAY peptide was described to have main contacts with residues G124, F192, Q201, V202, L203, V204, K210, V211, A212, V213, D215, A216, L218, L219, and Q220. According to our NMR data, the CTD residues that show significant chemical shifts in response to peptide3 binding are I132, V202, V204, E205, K210, V211, A212, V213, A216, L218 and L219 (FIGS. 7A-7B). These residues are all located within 5Δ of the predicted binding site of peptide3 according to the MD simulation. Thus the contacts made by the PGAY peptide and P3 have a large degree of overlap, but also some differences. However, majority of the residues that contact the PGAY peptide are located within 5Δ of peptide3, with the exception of G124 and Q220. This indicates that the gross binding sites of the two peptides are highly similar. In particular, I132, which is located in the (32 sheet of the CTD, is of interest because the adjacent mutation of L131 combined with L203 abrogated the agglutination of Galectin-3, suggesting that the (32 sheet may have a critical contribution to the IDR-CTD interaction.

A recent study by Zhao et al (21) provided insight into the important unresolved issue of the contribution of the NTD to oligomerization and liquid-liquid phase separation of Galectin-3. Their data provide evidence for a model in which the S-face of the CTD binds to glycoproteins and leaves the F-face to interact with the IDR of other Galectin-3 molecules as a key step in polymerization. The model proposes that the NTD-CTD interactions are in fact the primary driving force for Galectin-3-mediated, glycoprotein-dependent phase separation on the plasma membrane. Our finding that P3 efficiently inhibits Galectin-3 mediated agglutination of leukemia cells and binds the F-face of the CTD is consistent with their model (FIG. 1A).

In the current study we have addressed the question if it is possible to interfere with the interaction of an ITD with a domain of defined structure using Galectin-3 as a test case. Our results show that this is feasible. Because IDRs are enriched in many important proteins that form RNP complexes and membrane-less subcellular compartments such as stress granules, it may be possible to use a strategy similar to the one used here to disperse complexes in which they part of and inhibit their function.

While various embodiments and aspects of the disclosure are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments described herein may be employed.

### REFERENCES

Chan Y C, Lin H Y, Tu Z, Kuo Y H, Hsu S D, Lin C H. Dissecting the Structure-Activity Relationship of Galectin-Ligand Interactions. Int J Mol Sci. 2018; 19(2):392. Published 2018 Jan. 29. doi:10.3390/ijms19020392; Fei F, Joo E J, Tarighat S S, et al. B-cell precursor acute lymphoblastic leukemia and stromal cells communicate through Galectin-3. Oncotarget. 2015; 6(13):11378-11394. doi:10.18632/oncotarget.3409; Fei F, Abdel-Azim H, Lim M, et al. Galectin-3 in pre-B acute lymphoblastic leukemia. Leukemia. 2013; 27(12):2385-2388. doi:10.1038/1eu.2013.175; Hamelberg, D., Mongan, J., and McCammon, J. A. (2004). Accelerated molecular dynamics: a promising and efficient simulation method for biomolecules. J Chem Phys 120, 11919-11929; Han, B., Liu, Y. F., Ginzinger, S. W., and Wishart, D. S. (2011). SHIFTX2: significantly improved protein chemical shift prediction. J Biomol Nmr 50, 43-57; Ippel, H., Miller, M. C., Vertesy, S., Zheng, Y., Canada, F. J., Suylen, D., Umemoto, K., Romano, C., Hackeng, T., Tai, G., et al. (2016). Intra- and intermolecular interactions of human Galectin-3: assessment by full-assignment-based NMR. Glycobiology 26, 888-903; Paz H, Joo E J, Chou C H, et al. Treatment of B-cell precursor acute lymphoblastic leukemia with the Galectin-1 inhibitor PTX008. J Exp Clin Cancer Res. 2018; 37(1):67. Published 2018 Mar. 27. doi:10.1186/s13046-018-0721-7; St-Gelais J, Denavit V, Giguere D. Efficient synthesis of a galectin inhibitor clinical candidate (TD139) using a Payne rearrangement/azidation reaction cascade [published online ahead of print, 2020 May 13]. Org Biomol Chem. 2020; 10.1039/d0ob00910e. doi:10.1039/d0oboo910e.

### REFERENCES FOR BACKGROUND AND EXAMPLE 2

1. Wright P E, Dyson H J. Intrinsically disordered proteins in cellular signalling and regulation. Nat Rev Mol Cell Biol. 2015; 16(1):18-29.
2. Afanasyeva A, Bockwoldt M, Cooney C R, Heiland I, Gossmann T I. Human long intrinsically disordered protein regions are frequent targets of positive selection. Genome Res. 2018; 28(7):975-82.
3. Coppin L, Jannin A, Ait Yahya E, Thuillier C, Villenet C, Tardivel M, et al. Galectin-3 modulates epithelial cell adaptation to stress at the ER-mitochondria interface. Cell Death Dis. 2020; 11(5):360.
4. Magescas J, Sengmanivong L, Viau A, Mayeux A, Dang T, Burtin M, et al. Spindle pole cohesion requires glycosylation-mediated localization of NuMA. Sci Rep. 2017; 7(1):1474.
5. Jia J, Claude-Taupin A, Gu Y, Choi S W, Peters R, Bissa B, et al. Galectin-3 Coordinates a Cellular System for Lysosomal Repair and Removal. Dev Cell. 2020; 52(1):69-87 e8.
6. Coppin L, Leclerc J, Vincent A, Porchet N, Pigny P. Messenger RNA Life-Cycle in Cancer Cells: Emerging Role of Conventional and Non-Conventional RNA-Binding Proteins? Int J Mol Sci. 2018; 19(3).
7. Coppin L, Vincent A, Frenois F, Duchene B, Landaoui F, Stechly L, et al. Galectin-3 is a non-classic RNA binding protein that stabilizes the mucin MUC4 mRNA in the cytoplasm of cancer cells. Sci Rep. 2017; 7:43927.
8. Joeh E, O'Leary T, Li W, Hawkins R, Hung J R, Parker C G, et al. Mapping glycan-mediated Galectin-3 interactions by live cell proximity labeling. Proc Natl Acad Sci USA. 2020; 117(44):27329-38.
9. Sciacchitano S, Lavra L, Morgante A, Ulivieri A, Magi F, De Francesco G P, et al. Galectin-3: One Molecule for an Alphabet of Diseases, from A to Z. Int J Mol Sci. 2018; 19(2).
10. Suthahar N, Meijers W C, Sillje H H W, Ho J E, Liu F T, de Boer R A. Galectin-3 Activation and Inhibition in Heart Failure and Cardiovascular Disease: An Update. Theranostics. 2018; 8(3):593-609.
11. Farhadi S A, Liu R, Becker M W, Phelps E A, Hudalla G A. Physical tuning of Galectin-3 signaling. Proc Natl Acad Sci USA. 2021; 118(19).
12. Lee J J, Hsu Y C, Li Y S, Cheng S P. Galectin-3 Inhibitors Suppress Anoikis Resistance and Invasive Capacity in Thyroid Cancer Cells. Int J Endocrinol. 2021; 2021:5583491.
13. Dings R P M, Miller M C, Griffin R J, Mayo K H. Galectins as Molecular Targets for Therapeutic Intervention. Int J Mol Sci. 2018; 19(3).
14. Bertuzzi S, Quintana J I, Arda A, Gimeno A, Jimenez-Barbero J. Targeting Galectins With Glycomimetics. Front Chem. 2020; 8:593.
15. Blanchard H, Yu X, Collins P M, Burn-Erdene K. Galectin-3 inhibitors: a patent review (2008-present). Expert Opin Ther Pat. 2014; 24(10):1053-65.
16. Stegmayr J, Zetterberg F, Carlsson M C, Huang X, Sharma G, Kahl-Knutson B, et al. Extracellular and intracellular small-molecule Galectin-3 inhibitors. Sci Rep. 2019; 9(1):2186.
17. Hirani N, MacKinnon A C, Nicol L, Ford P, Schambye H, Pedersen A, et al. Target inhibition of Galectin-3 by inhaled TD139 in patients with idiopathic pulmonary fibrosis. Eur Respir J. 2021; 57(5).
18. Bratteby K, Torkelsson E, L′Estrade E T, Peterson K, Shalgunov V, Xiong M, et al. In Vivo Veritas: (18)F-Radiolabeled Glycomimetics Allow Insights into the Pharmacological Fate of Galectin-3 Inhibitors. J Med Chem. 2020; 63(2):747-55.
19. Smith B A H, Bertozzi C R. The clinical impact of glycobiology: targeting selectins, Siglecs and mammalian glycans. Nat Rev Drug Discov. 2021; 20(3):217-43.
20. Dumic J, Dabelic S, Flogel M. Galectin-3: an open-ended story. Biochim Biophys Acta. 2006; 1760(4):616-35.
21. Zhao Z, Xu X, Cheng H, Miller M C, He Z, Gu H, et al. Galectin-3 N-terminal tail prolines modulate cell activity and glycan-mediated oligomerization/phase separation. Proc Natl Acad Sci USA. 2021; 118(19).
22. Uchino Y, Woodward A M, Mauris J, Peterson K, Verma P, Nilsson U J, et al. Galectin-3 is an amplifier of the interleukin-1beta-mediated inflammatory response in corneal keratinocytes. Immunology. 2018; 154(3):490-9.
23. Mirandola L, Yu Y, Cannon M J, Jenkins M R, Rahman R L, Nguyen D D, et al. Galectin-3 inhibition suppresses drug resistance, motility, invasion and angiogenic potential in ovarian cancer. Gynecol Oncol. 2014; 135(3):573-9.
24. Mirandola L, Yu Y, Chui K, Jenkins M R, Cobos E, John C M, et al. Galectin-3C inhibits tumor growth and increases the anticancer activity of bortezomib in a murine model of human multiple myeloma. PLoS One. 2011; 6(7):e21811.
25. Ippel H, Miller M C, Vertesy S, Zheng Y, Canada F J, Suylen D, et al. Intra- and intermolecular interactions of human Galectin-3: assessment by full-assignment-based NMR. Glycobiology. 2016; 26(8):888-903.
26. Lin Y H, Qiu D C, Chang W H, Yeh Y Q, Jeng U S, Liu F T, et al. The intrinsically disordered N-terminal domain of Galectin-3 dynamically mediates multisite self-association of the protein through fuzzy interactions. J Biol Chem. 2017; 292(43):17845-56.
27. Chiu Y P, Sun Y C, Qiu D C, Lin Y H, Chen Y Q, Kuo J C, et al. Liquid-liquid phase separation and extracellular multivalent interactions in the tale of Galectin-3. Nat Commun. 2020; 11(1):1229.
28. Flores-Ibarra A, Vértesy S, Medrano F J, Gabius H J, Romero A. Crystallization of a human Galectin-3 variant with two ordered segments in the shortened N-terminal tail. Sci Rep. 2018; 8(1):9835.
29. Eswar N, John B, Mirkovic N, Fiser A, Ilyin V A, Pieper U, et al. Tools for comparative protein structure modeling and analysis. Nucleic Acids Res. 2003; 31(13):3375-80.
30. Nguyen H, Roe D R, Simmerling C. Improved Generalized Born Solvent Model Parameters for Protein Simulations. J Chem Theory Comput. 2013; 9(4):2020-34.
31. Robustelli P, Piana S, Shaw D E. Developing a molecular dynamics force field for both folded and disordered protein states. Proceedings of the National Academy of Sciences. 2018; 115(21):E4758-E66.
32. Hopkins C W, Le Grand S, Walker R C, Roitberg A E. Long-Time-Step Molecular Dynamics through Hydrogen Mass Repartitioning. Journal of Chemical Theory and Computation. 2015; 11(4):1864-74.
33. Salomon-Ferrer R, Gotz A W, Poole D, Le Grand S, Walker R C. Routine Microsecond Molecular Dynamics Simulations with AMBER on GPUs. 2. Explicit Solvent Particle Mesh Ewald. Journal of Chemical Theory and Computation. 2013; 9(9):3878-88.
34. Han B, Liu Y F, Ginzinger S W, Wishart D S. SHIFTX2: significantly improved protein chemical shift prediction. Journal of Biomolecular Nmr. 2011; 50(1):43-57.
35. Bottaro S, Bengtsen T, Lindorff-Larsen K. Integrating Molecular Simulation and Experimental Data: A Bayesian/Maximum Entropy Reweighting Approach. In: Gáspári Z, editor. Structural Bioinformatics: Methods and Protocols. New York, N.Y.: Springer US; 2020. p. 219-40.
36. Paz H, Joo E J, Chou C H, Fei F, Mayo K H, Abdel-Azim H, et al. Treatment of B-cell precursor acute lymphoblastic leukemia with the Galectin-1 inhibitor PTX008. J Exp Clin Cancer Res. 2018; 37(1):67.
37. George A A, Paz H, Fei F, Kirzner J, Kim Y M, Heisterkamp N, et al. Phosphoflow-Based Evaluation of Mek Inhibitors as Small-Molecule Therapeutics for B-Cell Precursor Acute Lymphoblastic Leukemia. PLoS One. 2015; 10(9): e0137917.
38. Fei F, Joo E J, Tarighat S S, Schiffer I, Paz H, Fabbri M, et al. B-cell precursor acute lymphoblastic leukemia and stromal cells communicate through Galectin-3. Oncotarget. 2015; 6(13):11378-94.
39. Umemoto K, Leffler H. Assignment of ¹H, ¹⁵N and 13C resonances of the C-terminal domain of human Galectin-3. J Biomol NMR. 2001; 20(1):91-2.
40. Zou J, Glinsky V V, Landon L A, Matthews L, Deutscher S L. Peptides specific to the Galectin-3 C-terminal domain inhibit metastasis-associated cancer cell adhesion. Carcinogenesis. 2005; 26(2):309-18.
41. Hamelberg D, Mongan J, McCammon J A. Accelerated molecular dynamics: a promising and efficient simulation method for biomolecules. J Chem Phys. 2004; 120(24):11919-29.
42. Fei F, Abdel-Azim H, Lim M, Arutyunyan A, von Itzstein M, Groffen J, et al. Galectin-3 in pre-B acute lymphoblastic leukemia. Leukemia. 2013; 27(12):2385-8.
43. Beard H, Cholleti A, Pearlman D, Sherman W, Loving K A. Applying physics-based scoring to calculate free energies of binding for single amino acid mutations in protein-protein complexes. PLoS One. 2013; 8(12): e82849.
44. Miller B R, McGee T D, Swails J M, Homeyer N, Gohlke H, Roitberg A E. MMPBSA.py: An Efficient Program for End-State Free Energy Calculations. Journal of Chemical Theory and Computation. 2012; 8(9):3314-21.
45. St-Gelais J, Denavit V, Giguere D. Efficient synthesis of a galectin inhibitor clinical candidate (TD139) using a Payne rearrangement/azidation reaction cascade. Org Biomol Chem. 2020; 18(20):3903-7.
46. Chan Y C, Lin H Y, Tu Z, Kuo Y H, Hsu S D, Lin C H. Dissecting the Structure-Activity Relationship of Galectin-Ligand Interactions. Int J Mol Sci. 2018; 19(2).
47. Hsieh T J, Lin H Y, Tu Z, Lin T C, Wu S C, Tseng Y Y, et al. Dual thio-digalactoside-binding modes of human galectins as the structural basis for the design of potent and selective inhibitors. Sci Rep. 2016; 6:29457.
48. Miller M C, Zheng Y, Suylen D, Ippel H, Canada F J, Berbis M A, et al. Targeting the CRD F-face of Human Galectin-3 and Allosterically Modulating Glycan Binding by Angiostatic PTX008 and a Structurally Optimized Derivative. ChemMedChem. 2021; 16(4):713-23.
49. Zhang Z, Miller M C, Xu X, Song C, Zhang F, Zheng Y, et al. NMR-based insight into Galectin-3 binding to endothelial cell adhesion molecule CD146: Evidence for noncanonical interactions with the lectin's CRD beta-sandwich F-face. Glycobiology. 2019; 29(8):608-18.
50. Uversky V N. Intrinsic disorder-based protein interactions and their modulators. Curr Pharm Des. 2013; 19(23):4191-213.
51. Bhattacharya S, Lin X. Recent Advances in Computational Protocols Addressing Intrinsically Disordered Proteins. Biomolecules. 2019; 9(4).
52. Joshi P, Vendruscolo M. Druggability of Intrinsically Disordered Proteins. Adv Exp Med Biol. 2015; 870:383-400.
53. Uversky V N. Intrinsically Disordered Proteins. Structural Biology in Drug Discovery 2020. p. 587-612.
54. Cheng Y, LeGall T, Oldfield C J, Mueller J P, Van Y Y, Romero P, et al. Rational drug design via intrinsically disordered protein. Trends Biotechnol. 2006; 24(10):435-42.
55. Navarro P, Martinez-Bosch N, Blidner A G, Rabinovich G A. Impact of Galectins in Resistance to Anticancer Therapies. Clin Cancer Res. 2020; 26(23):6086-101.
56. Girotti M R, Salatino M, Dalotto-Moreno T, Rabinovich G A. Sweetening the hallmarks of cancer: Galectins as multifunctional mediators of tumor progression. J Exp Med. 2020; 217(2).
57. Zerbe B S, Hall D R, Vajda S, Whiny A, Kozakov D. Relationship between hot spot residues and ligand binding hot spots in protein-protein interfaces. J Chem Inf Model. 2012; 52(8):2236-44.
58. Stasenko M, Smith E, Yeku O, Park K J, Laster I, Lee K, et al. Targeting Galectin-3 with a high-affinity antibody for inhibition of high-grade serous ovarian cancer and other MUC16/CA-125-expressing malignancies. Sci Rep. 2021; 11(1):3718.
59. Miller M C, Ippel H, Suylen D, Klyosov A A, Traber P G, Hackeng T, et al. Binding of polysaccharides to human Galectin-3 at a noncanonical site in its C-terminal domain. Glycobiology. 2016; 26(1):88-99.
60. Miller M C, Klyosov A A, Mayo K H. Structural features for alpha-galactomannan binding to galectin-1. Glycobiology. 2012; 22(4):543-51.

What is claimed is:

1. A method of identifying an amino acid within a disordered domain of a protein that binds to an ordered domain of a protein with the ordered domain either located in the same protein or in a different protein, the method comprising:
(i) in silico, performing an enhanced sampling of a disordered domain of a protein binding to an ordered domain of the same protein or an ordered domain of a different protein thereby obtaining an ensemble of conformations, wherein each conformation in the ensemble comprises the disordered domain bound to the ordered domain;
(ii) identifying a first set of structural conformations from the ensemble of conformations that satisfy the experimental structural NMR data or small angle X-ray scattering data of the protein; and
(iii) identifying a first amino acid within the first set of structural conformations, wherein the first amino acid is within the disordered domain of the protein that binds to the ordered domain of the same protein or binds to the ordered domain of the different protein.

2. The method of claim 1, wherein step (ii) comprises identifying the first set of structural conformations from the ensemble of conformations that satisfy the experimental structural NMR data of the protein

3. The method of claim 1 or 2, further comprising: (iv) clustering the first set of structural conformations by structural similarity to identify template peptides.

4. The method of any one of claims 1 to 3, further comprising identifying a second amino acid within the first set of structural confirmations, wherein the second amino acid is within the ordered domain of the same or different protein that binds to the disordered domain of the protein.

5. The method of any one of claims 1 to 4, wherein the first amino acid within the first set of structural conformations comprises at least two amino acids.

6. The method of any one of claims 1 to 5, wherein the enhanced sampling simulation comprises accelerated molecular dynamic simulations.

7. The method of any one of claims 1 to 5, wherein the enhanced sampling simulation comprises molecular dynamics, Monte Carlo, replica exchange molecular dynamics simulation, metadynamics simulation, temperature cool walking, or generalized simulated annealing.

8. The method of any one of claims 1 to 7, further comprising:
(a) designing a plurality of template peptides that bind in silico to at least one amino acid in the ordered domain based at least in part on the first set of structural conformations;
(b) in silico, mutating each amino acid residue of each of the plurality of template peptides thereby producing a plurality of mutant peptides;
(c) selecting a set of candidate peptides from the plurality of mutant peptides based on in silico binding;
(d) synthesizing each of the set of candidate peptides thereby producing a set of synthesized candidate peptides; and
(e) experimentally measuring the effect of each of the synthesized candidate peptides on a protein.

9. The method of claim 8, wherein the effect in (e) is binding.

10. A compound capable of inhibiting an interaction between a disordered N-terminal domain of Galectin-3 and an allosteric cavity in a C-terminal domain of Galectin-3.

11. The compound of claim 10, wherein the allosteric cavity in the C-terminal domain of Galectin-3 is a F-face of the C-terminal domain of Galectin-3.

12. The compound of claim 10 or 11, wherein the compound is capable of inhibiting an interaction between at least one amino acid in the disordered N-terminal domain of Galectin-3 and at least one amino acid in the allosteric cavity in the C-terminal domain of Galectin-3.

13. The compound of claim 12, wherein the at least one amino acid in the disordered N-terminal domain of Galectin-3 is selected from the group consisting of A2, A49, A53, A69, D3, F5, G108, G112, G43, G47, G52, G68, G72, H8, P106, P71, Q20, Q48, S84, T98, V78, W22, Y101, Y41, Y36, Y45, Y54, Y70, Y79, T104, Y89, and A100.

14. The compound of claim 12 or 13, wherein the at least one amino acid in the allosteric cavity in the C-terminal domain of Galectin-3 is selected from the group consisting of Y247, T243, Q201, V202, K210, A216, F192, F198, K199, L203, V204, D215, H217, Q220, L219, L131, V211, A212, V213, L218, E205, and I132.

15. The compound of any one of claims 10 to 12, wherein the compound is capable of inhibiting an interaction between Y36 and/or Y45 in the disordered N-terminal domain of Galectin-3 and the allosteric cavity in the C-terminal domain of Galectin-3.

16. The compound of any one of claims 10 to 12, wherein the compound is capable of inhibiting an interaction between Y36 in the disordered N-terminal domain of Galectin-3 and V202, K210, A216, F192, F198, K199, L203, V204, D215, H217, Q220, L219, or a combination of two or more thereof in the allosteric cavity in the C-terminal domain of Galectin-3.

17. The compound of any one of claims 10 to 12, wherein the compound is capable of inhibiting an interaction between Y45 in the disordered N-terminal domain of Galectin-3 and V202, K210, A216, F192, F198, K199, L203, V204, D215, H217, Q220, L219, or a combination of two or more thereof in the allosteric cavity in the C-terminal domain of Galectin-3.

18. The compound of any one of claims 10 to 12, wherein the compound is capable of inhibiting an interaction between Y36 in the disordered N-terminal domain of Galectin-3 and V202, K210, A216, or a combination of two or more thereof in the allosteric cavity in the C-terminal domain of Galectin-3.

19. The compound of any one of claims 10 to 12, wherein the compound is capable of inhibiting an interaction between Y45 in the disordered N-terminal domain of Galectin-3 and V202, K210, A216, or a combination of two or more thereof in the allosteric cavity in the C-terminal domain of Galectin-3.

20. The compound of any one of claims any one of claims 10 to 19, wherein the compound is a peptide, a small molecule, or a macrocycle.

21. The compound of any one of claims 10 to 20, wherein the compound has an inhibitory effect on Galectin-3 that is the same as or better than the peptide comprising the amino acid sequence of SEQ ID NO:9 and/or that fills the same space as the peptide comprising the amino acid sequence of SEQ ID NO:9.

22. The compound of any one of claims 10 to 20, wherein the compound is a peptide comprising SEQ ID NO:3.

23. The compound of any one of claims 10 to 20, wherein the compound is a peptide comprising SEQ ID NO:9.

24. The compound of any one of claims 10 to 23, wherein the compound is covalently bonded to (i) a delivery agent, (ii) a detectable agent, or (iii) a delivery agent and a detectable agent.

25. The compound of claim 24, wherein the delivery agent comprises a polymer or a copolymer.

26. The compound of claim 24 or 25, wherein the detectable agent is a radioactive agent, a fluorescent agent, a phosphorescent agent or a luminescent agent.

27. A pharmaceutical composition comprising the compound of any one of claims 10 to 26 and a pharmaceutically acceptable excipient.

28. A method for treating cancer in a subject in need thereof, the method comprising administering to the subject an effective amount of the compound of any one of claims 10 to 26, or the pharmaceutical composition of claim 27.

29. A method for detecting cancer in a subject in need thereof, the method comprising administering to the subject an effective amount of the compound of any one of claims 10 to 26, or the pharmaceutical composition of claim 27; and detecting the detectable agent in the human.

30. The method of claim 28 or 29, wherein the cancer overexpresses or inappropriately expresses Galectin-3.

31. The method of any one of claims 28 to 30, wherein the cancer is leukemia.

32. The method of claim 31, wherein the leukemia is acute lymphoblastic leukemia.

33. The method of any one of claims 28 to 30, wherein the cancer is ovarian cancer, breast cancer, bladder cancer, gastric cancer, prostate cancer, lung cancer, pancreatic cancer, thyroid cancer, colon cancer, melanoma, or lymphoma.

34. A method for treating fibrosis in a subject in need thereof, the method comprising administering to the subject an effective amount of the compound of any one of claims 10 to 26, or the pharmaceutical composition of claim 27.

35. A method for treating a cardiovascular disease in a subject in need thereof, the method comprising administering to the subject an effective amount of the compound of any one of claims 10 to 26, or the pharmaceutical composition of claim 27.

36. A method for treating an infectious disease in a subject in need thereof, the method comprising administering to the subject an effective amount of the compound of any one of claims 10 to 26, or the pharmaceutical composition of claim 27.

37. A method for treating an inflammatory disease in a subject in need thereof, the method comprising administering to the subject an effective amount of the compound of any one of claims 10 to 26, or the pharmaceutical composition of claim 27.

38. A method for treating a neurological disease in a subject in need thereof, the method comprising administering to the subject an effective amount of the compound of any one of claims 10 to 26, or the pharmaceutical composition of claim 27.

39. A method for inhibiting a Galectin-3 protein, the method comprising contacting the compound of any one of claims 10 to 26, or the pharmaceutical composition of claim 27 with the Galectin-3 protein; thereby inhibiting the Galectin-3 protein.

40. A method for treating a disease characterized by overexpression or inappropriate expression of Galectin-3 in a subject in need thereof, the method comprising administering to the subject an effective amount of the compound of any one of claims 10 to 26, or the pharmaceutical composition of claim 27.

41. A system comprising at least one data processor and at least one memory storing instructions which, when executed by the at least one data processor, result in operations comprising identifying an amino acid within a disordered domain of a protein that binds to an ordered domain of a protein with the ordered domain either located in the same protein or in a different protein as set forth in any one of claims 1 to 9.

42. A computer-implemented method, the method comprising identifying an amino acid within a disordered domain of a protein that binds to an ordered domain of a protein with the ordered domain either located in the same protein or in a different protein as set forth in any one of claims 1 to 9.

43. A non-transitory computer readable medium storing instructions, which when executed by at least one data processor, result in operations comprising identifying an amino acid within a disordered domain of a protein that binds to an ordered domain of a protein with the ordered domain either located in the same protein or in a different protein as set forth in any one of claims 1 to 9.
